(19) United States
(12) Patent Application Publication
(10) Pub. No.: US 2026/0177567 A1
(43) Pub. Date: Jun. 25, 2026

(54) METHODS RELATED TO BIOLOGICAL AGE USING METABOLITE BIOMARKERS

(71) Applicant: Société des Produits Nestlé S.A., vevey (CH)

(72) Inventors: Rondo P MIDDLETON, CREVE COEUR, MO (US); Pan Yuanlong, Chesterfield, MO (US); Chang Woon Jang, Chesterbrook, PA (US)

(73) Assignee: Société des Produits Nestlé S.A., vevey (CH)

(21) Appl. No.: 19/368,567

(22) Filed: Oct. 24, 2025

Related U.S. Application Data
(60) Provisional application No. 63/736,189, filed on Dec. 19, 2024.

(51) Int. Cl. G01N 33/92

(52) U.S. Cl. CPC G01N 33/92; G01N 2570/00; G01N 2800/52; G01N 2800/7042

(57) ABSTRACT

The present invention comprises a method for determining the biological age of an animal, wherein the method comprises determining the level of at least one biomarker in said animal, and wherein the at least 1 biomarker is selected from the biomarkers as listed in Table 1.

### CROSS REFERENCE TO RELATED APPLICATION

This application claims priority to U.S. Provisional Application Ser. No. 63/736,189 filed Dec. 19, 2024 the disclosure of which is incorporated in its entirety herein by this reference.

### FIELD OF THE INVENTION

The present invention relates to a method for determining the biological age of a subject using levels of biomarkers in said subject. In particular, the present invention relates to a method for determining the biological age of a dog using levels of metabolite biomarkers in the dog. The invention further relates to methods of selecting a dietary, pharmacological or lifestyle regime for a subject, for example to provide a suitable dietary, pharmacological or lifestyle regime appropriate to the subject's biological age.

### BACKGROUND TO THE INVENTION

The ability to determine information regarding the health of an animal is desirable to inform about the animal's general health and well-being.

Chronological age is known to be a major indicator of general health status, with increasing chronological age associated with reduced health. However, depending on genetics, nutrition, and lifestyles, individuals may age slower or faster than their chronological age. Chronological age may therefore not always reflect an individual's rate of aging or risk of reduced health. On the other hand, the biological age of an individual (based on e.g. clinical biochemistry and cell biology measures) can vary compared to others of the same chronological age. Methods for determining biological age may be helpful for identifying individuals at risk of age-related disorders earlier than would be expected based on their chronological age.

However, there is a need for further methods of determining the biological age of an animal and utilising measures of biological age to improve health outcomes for an animal.

### SUMMARY OF THE INVENTION

In a first aspect, the present invention provides a method of determining the biological age of an animal subject, wherein the method comprises determining the levels of at least one biomarker in said animal, wherein the at least 1 biomarker is selected from the biomarkers as listed in Table 1.

Suitably, the method comprises determining the levels of five or more biomarkers in said animal, wherein at least 1 biomarker is selected from the biomarkers as listed in Table 1.

The present invention provides markers that allow the biological age of an animal subject to be determined. Advantageously, the determination of the biological age of an animal subject may be used to select a suitable dietary, pharmacological or lifestyle regime for the subject.

In one aspect, the present invention provides a method for selecting a dietary, pharmacological or lifestyle regime for an animal subject based on the determined biological age of the subject.

As used herein, ‘selecting a dietary, pharmacological or lifestyle regime for an animal subject’ may also encompass ‘recommending a dietary, pharmacological or lifestyle regime for the animal subject’ or ‘providing a recommended dietary, pharmacological or lifestyle regime for the animal subject’.

In a further aspect, the present invention provides a method for determining the efficacy of a dietary, pharmacological or lifestyle regime for improving the biological age of an animal subject, wherein the biological age of the subject is determined from a sample obtained from the subject before the dietary, pharmacological or lifestyle regime, the biological age of the subject is determined from a samples obtained from the subject after the dietary, pharmacological or lifestyle regime, and it is determined if there has been a change in the biological age of the subject before and after the dietary, pharmacological or lifestyle regime has been applied.

In a further aspect, the present invention provides a method for determining a likelihood that an animal subject will benefit from a dietary, pharmacological or lifestyle regime for improving the biological age of an animal subject, wherein the biological age of the subject is determined, and the subject is identified as being likely to benefit from a dietary, pharmacological or lifestyle regime for improving the biological age of an animal subject if it has an increased biological age relative to its chronological age or it has an biological age equal to its chronological age.

In a further aspect, the present invention provides a method of treating, preventing or reducing the risk of an age-related disease in an animal subject, wherein the method comprises determining the biological age of the subject, and administering a dietary, pharmacological or lifestyle regime to the subject.

For example, the age-related disease may be osteoarthritis, dementia, cognitive dysfunction, pre-diabetic condition, diabetes, cancer, heart disease, obesity, gastrointestinal disorders, incontinence, kidney disease, sarcopenia, frailty, vision loss, hearing loss, osteoporosis, cataracts, wrinkles, itching and dry skin, cerebrovascular disease, liver disease, and/or an immune system or immune-related disease or disorder.

In one aspect, the present invention provides a method of reducing the risk of an age-related disease in an animal subject, wherein the method comprises determining the biological age of the subject, and administering a dietary, pharmacological or lifestyle regime to the subject.

In one aspect, the present invention provides a dietary or pharmaceutical product for use in treating, preventing or reducing the risk of an age-related disease in an animal subject, wherein the dietary or pharmaceutical product is administered to a subject for which a biological age has been determined.

In one aspect, the present invention provides a dietary or pharmaceutical product for use in reducing the risk of an age-related disease in an animal subject, wherein the dietary or pharmaceutical product is administered to a subject for which a biological age has been determined.

In one aspect, the present invention provides the use of a dietary or pharmaceutical product for the manufacture of a medicament for the treatment, prevention or reduction of the risk of an age-related disease in an animal subject, wherein the medicament is administered to a subject for which a biological age has been determined.

In one aspect, the present invention provides the use of a dietary or pharmaceutical product for the manufacture of a medicament for the reduction of the risk of an age-related disease in an animal subject, wherein the medicament is administered to a subject for which a biological age has been determined.

In some embodiments, the method of treating, preventing or reducing the risk of an age-related disease is applied to a subject determined to be at risk of suffering from an age-related disease. In some embodiments, the dietary or pharmaceutical product for use in treating, preventing or reducing the risk of an age-related disease is administered to a subject determined to be at risk of suffering from an age-related disease.

In some embodiments, the risk of an animal subject suffering from an age-related disease is identified based on the determined biological age. In some embodiments, the subject may be considered at risk of suffering from an age-related disease where the subject is determined to have an increased biological age relative to its chronological age. In some embodiments, the subject may be considered at risk of suffering from an age-related disease where the subject is determined to have a biological age equal to its chronological age. In some embodiments, the subject may be considered at risk of suffering from an age-related disease wherein the subject is determined to be in a particular biological age category.

Suitably, the present methods comprise providing the levels of biomarkers in an animal subject from a sample obtained from the subject.

Suitably, the biomarkers are metabolites.

The present invention further provides a computer-readable medium comprising instructions that when executed cause one or more processors to perform the method of the present invention.

The invention also provides a computer system for selecting a dietary, pharmacological or lifestyle regime for an animal subject, the computer system programmed to perform the steps of (i) determining the biological age of the subject, wherein the biological age is determined by the method of the present invention and (ii) selecting a suitable dietary, pharmacological, or lifestyle regime for the subject based on the determined biological age.

The invention also provides a computer system for determining the efficacy of a dietary, pharmacological or lifestyle regime for improving the biological age of an animal subject, the computer system programmed to perform the steps of (i) determining the biological age of the subject, wherein the biological age is determined by the method of the present invention from a sample obtained from the subject before administration of the dietary, pharmacological or lifestyle regime, (ii) determining the biological age of the subject, wherein the biological age is determined by the method of the present invention from a sample obtained from the subject after administration of the dietary, pharmacological or lifestyle regime, and (iii) comparing the determined biological ages from before and after administration of the dietary, pharmacological or lifestyle regime and determining if there has been a change in the biological age of the subject between the sample obtained from the subject before and after the dietary, pharmacological or lifestyle regime has been applied.

The invention also provides a computer system for determining a likelihood that an animal subject will benefit from a dietary, pharmacological or lifestyle regime for improving the biological age of an animal subject, the computer system programmed to perform the steps of (i) determining the biological age of the subject, wherein the biological age is determined by the method of the present invention and (ii) identifying a subject as likely to benefit from a dietary, pharmacological or lifestyle regime for improving the biological age of an animal subject if it has an increased biological age relative to its chronological age or if it has a biological age equal to its chronological age.

The invention also provides a computer system for selecting a dietary, pharmacological or lifestyle regime for an animal subject, the computer system programmed to perform the steps of (i) determining the biological age of the subject, wherein the biological age is determined by the method of the present invention, (ii) determining whether the subject is at risk of age-related disease, wherein the risk of age-related disease is determined based on the biological age of the subject and/or the comparison of the determined biological age and the chronological age, and (iii) selecting a suitable dietary, pharmacological, or lifestyle regime for the subject based on the determined risk of age-related disease.

The invention also provides a computer program product comprising computer implementable instructions for causing a programmable computer to (i) determine the biological age of an animal subject, wherein the biological age is determined by the method of the present invention, and (ii) select a suitable dietary, pharmacological, or lifestyle regime for the subject based on determined biological age and/or a comparison of the determined biological age and the chronological age.

The invention further provides a computer program product comprising computer implementable instructions for causing a programmable computer to (i) determine the biological age of an animal subject, wherein the biological age is determined by the method of the present invention, and wherein the biological age is determined from a sample obtained from the subject before a dietary, pharmacological or lifestyle regime has been applied to the subject, (ii) determine the biological age of the subject, wherein the biological age is determined by the method of the present invention, and wherein the biological age is determined from a sample obtained from the subject after a dietary, pharmacological or lifestyle regime has been applied to the subject, (iii) determine if there has been a change in the biological age of the subject between the sample obtained from the subject before and after the dietary, pharmacological or lifestyle regime has been applied, and (iv) determine the efficacy of the dietary, pharmacological or lifestyle regime in improving the biological age of the animal subject, wherein a reduction in the biological age of the subject indicates efficacy of the dietary, pharmacological or lifestyle regime.

### DESCRIPTION OF THE DRAWINGS

FIG. 1—Ages of animals in the dataset. Age given is the chronological age of the dog at sample collection. Dogs were split into groups based on their chronological age. A young group comprised 2-5 year-old dogs, and a senior group comprised 7+ year old dogs.

FIG. 2—Solid phase extraction protocol for extraction of polar metabolites and lipids from plasma samples by LC/MS.

FIG. 3—An AdaBoost regressor was trained on a training dog metabolomics dataset and optimal hyperparameters set. The AdaBoost regression model showed an r² value of 0.39 for the testing set with the optimal hyperparameters obtained from the grid search with 5-fold cross-validation (dot: training set, x: testing set).

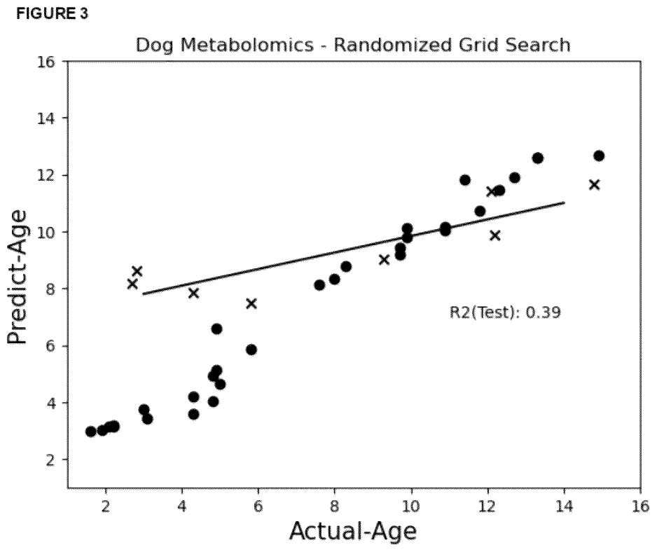

FIG. 4—Identification of the top 35 most important features in the AdaBoost Regressor model with their feature importance. Metabolites are plotted in order of feature importance (bottom to top).

FIG. 5—Models were constructed using varying numbers of features. The r² value of the models is plotted against the number of features used in the model. The model using the top 35 most important features had an r² value of 0.69. The solid horizontal line indicates the reference r-squared value of 0.39 from the model containing all features.

FIG. 6—Ridge regression models were constructed with varying numbers of features. The r-squared (r²) value is plotted against the number of features used in the model. The ridge regression model reaches an r² value of 0.45 with the top 20 important features, and 0.38 with the top 35 most important features. The solid horizontal line indicates the reference r-squared value of 0.39 from the Adaboost regression model containing all features.

FIG. 7—SVM classification plot for dog metabolomics. It shows 100% accuracy to classify young and senior dogs.

FIG. 8—The categorical model accuracy was plotted as a function of the number of important metabolites used in the model. The accuracy of the model reaches 1.0 when using only the top 8 most important features

### DETAILED DESCRIPTION

### Subject

The present methods are directed to animal subjects. Suitably, the animal subject of the present invention is a mammal. In some embodiments of the invention, the subject is a canine. In some embodiments of the invention, the subject is a dog, suitably a domestic dog.

The present methods may utilise information regarding the breed of the dog. The dog may be categorised as a toy, small, medium, large or giant breed—for example. Suitably, the dog breed may be categorised based on the weight of the dog. Suitably, the dog breed may be categorised based on the average weight of a dog for a given breed.

Suitably, the dog may be categorised as a toy breed, small or medium breed, or large breed, or giant breed. Suitably, the categorisation is determined by the average weight of adult dogs of this breed. Suitably, a breed with an average weight between 2.2 kg and 5.5 kg is categorised as a toy breed, a breed with an average weight between 5.5 kg and 10 kg is categorised as a small breed and/or a breed with an average weight between 10 kg and 25 kg is categorised as a medium breed. A breed with an average weight between 26 kg and 45 kg is categorised as large breed and a breed with an average weight above 45 kg is categorised as giant breed

Suitably, the sex of the animal may be classified as male or female.

### Chronological Age

Chronological age may be defined as the amount of time that has passed from the subject's birth to the given date. Chronological age may be expressed in terms of years, months, days, etc.

Suitably, the present method may be applied to a subject of any chronological age. In certain embodiments, the animal subject is a dog wherein the dog may be at least about 2 years old. Suitably, the dog may be at least about 2, at least about 3, at least about 4, at least about 5, at least about 6, at least about 7, at least about 8, at least about 9 or at least about 10 years old or older.

### Biological Age

Depending on genetics, nutrition, and lifestyles—for example—individuals may age slower or faster than their chronological age. Chronological age may therefore not always reflect an individual's biological rate of aging. As such, the biological age of an individual (based on e.g. clinical biochemistry and cell biology measures) can vary compared to others of the same chronological age.

The biological age of an individual is representative of the functional status of the cells and tissues of the individual, and how much an individual's body has been affected by ageing processes. The biological age of an individual can be useful to predict a risk of age-related diseases or conditions, as well as mortality risk.

The methods of the present invention determine the biological age of an animal. In some embodiments, the methods of the present invention determine the numerical biological age of an animal. Suitably, the numerical biological age is a continuous variable. The numerical biological age of an animal may be determined in terms of any suitable unit of time. Suitably, the numerical biological age of an animal is determined in terms of years or months. In some embodiments, the methods of the present invention determine a categorical biological age of an animal. Suitably, the categorical biological age is determined in terms of age categories based on age ranges of any suitable unit of time. Suitably, biological age categories may include numerical ranges in terms of years or months. In some embodiments, the categorical biological age is determined between two or more categories. Suitably, the categories of biological age may include categories such as junior, senior, infant, adult, geriatric, or mature. In some embodiments, wherein the animal subject is a domestic dog, the categories of biological age may include puppy, junior, adult, senior or mature. In some embodiments, wherein the animal subject is a domestic dog, the categories of biological age comprise junior, adult and senior. In some embodiments, wherein the animal subject is a domestic dog, the categories of biological age comprise junior and senior. Suitably, wherein the animal subject is a domestic dog, the categories of biological age are junior and senior, wherein junior includes biological ages of 2 years to less than 7 years, and senior includes biological ages of 7+ years (i.e. 7 or more years).

Suitably, the methods of the present invention may be applied to an animal subject of any biological or chronological age.

### Sample

The present invention may comprise a step of determining the level of one or more biomarker from one or more sample obtained from an animal subject.

The present invention may comprise a step of determining the levels of at least one, preferably five or more, biomarkers from one or more sample obtained from an animal subject. In some embodiments, the levels of biomarkers are determined from a single sample obtained from an animal subject.

Suitably, the one or more sample may be a sample of any suitable biological material obtained from an animal subject. Suitably, the one or more sample may be a blood, urine, faecal, saliva, hair follicle, buccal swab, or tissue sample.

Suitably, the sample is derived from blood. The sample may contain a blood fraction or may be whole blood. In some embodiments, the sample is a serum sample. In some embodiments, the sample is a plasma sample.

### Biomarkers

The present invention comprises determining the level of biomarkers in an animal subject. Suitably, the biomarkers are metabolites.

Suitably, the methods of the present invention comprise determining the level of one or more metabolite in an animal. Suitably, the level of one or more metabolite in an animal is determined from a sample obtained from said animal.

Suitably, the methods of the present invention comprise determining the level of one or more metabolite in the serum of an animal. Suitably, the level of one of more metabolite in the serum of an animal is determined from a serum sample obtained from said animal. Suitably, the methods of the present invention comprise determining the level of one or more metabolite in the plasma of an animal. Suitably, the level of one of more metabolite in the plasma of an animal is determined from a plasma sample obtained from said animal

Determining the level of a metabolite in an animal may be carried out using any suitable method for determining metabolite levels known in the art. Suitably, the term metabolomics may be used to refer to methods of determining metabolite levels, and the term lipidomics may be used to refer to methods of determining more specifically hydrophobic metabolites, such as lipids.

### Metabolites

A metabolite may be a small molecule associated with cell metabolism. Suitably, the metabolite may be a substrate, product or intermediate of cellular metabolism. Suitably, the metabolite may be a hydrophobic metabolite. Suitably, the metabolite may be a polar metabolite. For example, the polar metabolite may be an amino acid. The hydrophobic metabolite may be a fatty acid molecule.

Suitably, the metabolite may be specified using a metabolite or lipid name, or a database accession number. For example, the metabolite may be specified using a Human Metabolome Database (HMDB), Kyoto Encyclopedia of Genes and Genomes (KEGG), PubChem or Chemical Entities of Biological Interest (ChEBI) identification number.

Both the metabolite names and database accession number would be well known to, and understood by, the person of skill in the art. In some embodiments, the metabolite may be common to all animals of a certain taxonomic rank. Suitably the metabolite may be common to all animals of the class mammalia. In some embodiments the metabolite may be species specific.

Suitably, the metabolite may be a lipid. Forms of lipid nomenclature are known and understood by the person of skill in the art. Suitably, lipid metabolites may be identified using a common name, using shorthand notation at the species level, using shorthand notation at the bond type level, using shorthand notation at the fatty acyl/alkyl level, using shorthand notation at the fatty acyl/alkyl position level or the LIPID MAPS Fatty acyl/alkyl structure level. Preferably, lipid metabolites may be identified using shorthand notation at the fatty acyl/alkyl level or shorthand notation at the bond type level. Shorthand at the fatty acyl/alkyl level identifies the lipid class by abbreviation, and identifies the number of carbon atoms and double-bonds for each chain. Lipid class abbreviations include DG for diglycerides, TG for triglycerides, PG for phosphatidylgylcerols, PC for phosphatidylcholine, LPC for lysophosphatidylcholines, PE for phosphatidylethanolamine, PS for phosphatidylserine, PI for phosphatidylinositol, CE for cholesteryl esters, SM for sphingomyelins, HexCer for hexosylceramides and ACar for acylcarnitines. As an example, DG 20:1_18:2 refers to a diglyceride with one fatty acid chain containing 20 carbons and one carbon-carbon double bond, and a second fatty acid chain containing 18 carbons and two carbon-carbon double bonds. Shorthand at the bond type level identifies the lipid class by abbreviation, and identifies the number of carbon atoms and double-bonds in the lipid. Lipid class abbreviations include DG for diglycerides, TG for triglycerides, PG for phosphatidylgylcerols, PC for phosphatidylcholine, LPC for lysophosphatidylcholines, PE for phosphatidylethanolamine, PS for phosphatidylserine, PI for phosphatidylinositol, CE for cholesteryl esters, SM for sphingomyelins, HexCer for hexosylceramides and ACar for acylcarnitines. As an example, TG 54:8 refers to a triglyceride with 54 carbon atoms and 8 carbon-carbon double bonds. Where a given specificity of nomenclature is used, it is intended that the metabolites are distinguished at that level of specificity of nomenclature.

Exemplary metabolites that have alternate standardized lipid names or HMDB IDs are provided below:

| | HMDB ID/alternate |
|---|---|
| | standardized lipid |
| Metabolites | name |
| L-Carnitine | HMDB0000062 |
| DG 20:1_18:2 | DG 18:2_20:1 |
| N-Acetylleucine | HMDB0011756 |
| 4-Acetamidobutanoate | HMDB0003681 |
| ACar 18:0 | CAR 18:0 |
| Isocitrate | HMDB0000193 |
| Citrate | HMDB0000094 |
| SM d41:1 | SM 41:1; O2 |
| 1-Phenylethanol | HMDB0032619 |
| 10-Hydroxydecanoate | HMDB0244272 |
| HexCer_NS d18:1_16:0 | HexCer 18:1; O2/16:0 |
| N-Acetylputrescine | HMDB0002064 |
| Leucine | HMDB0000687 |
| Phenylalanine | HMDB0000159 |
| Isoleucine | HMDB0000172 |
| 4-Hydroxybenzoate | HMDB0304180 |

Suitably, the level of a metabolite in a subject is given by the abundance of a metabolite quantified in a sample.

Suitably, the levels of some or all of the metabolites in Table 1, Table 2 and/or Table 3 are determined by any method of metabolite quantification known in the art. Suitable, exemplary methods are described below.

Suitably, simultaneous quantification of a large number of metabolites can be achieved via mass-spectrometry (MS) or nuclear magnetic resonance (NMR) techniques.

### Mass-Spectrometry (MS) Techniques

MS techniques may be used to identify metabolites based on mass to charge ratio m/z values. Direct injection mass spectrometry approaches are able to differentiate and quantify metabolites with high mass-resolution. Direct injection approaches are unable to differentiate isobars (compounds with the same nominal mass but very slightly different exact masses). Suitably, a liquid- or gas-chromatography step may be carried out prior to mass spectrometry detection (LC-MS or GC-MS) to enable separation of isobars. For detection of polar metabolites and lipids from the same sample, a two-phase extraction process allows extraction of these species separately, followed by (LC/GC)-MS of each phase independently.

### Nuclear Magnetic Resonance Techniques

NMR techniques may be used to identify metabolites based on spectroscopic properties of metabolites. NMR is also able to quantify metabolite abundance (qNMR) due to the NMR signal being proportional to the abundance of a particular nuclei forming part of a metabolite's chemical structure. NMR is particularly advantageous in gathering structural determination of metabolites, and hence is able to differentiate metabolites by structure easily, though can be less sensitive to lower-abundance molecules, due to higher abundance signal peaks obscuring the lower strength peaks of lower-abundance metabolites.

Illustrative methods for identifying metabolites are given in the examples. For example, MS/MS for both polar metabolites and lipids may be acquired using commercially available software e.g. MassHunter Acquisition Software (Version 10.1.48, Agilent Technologies) and using commercially available instruments e.g. an Agilent 6540 or 6545 QTOF. The person of skill in the art would be able to identify polar metabolites by, for example, matching retention time, accurate mass and MS/MS fragmentation data to e.g. libraries created from authentic reference standards (such as Mass Spectrometry Metabolite Library supplied by IROA Technologies, Millipore Sigma, St. Louis, MO, USA) and online MS/MS libraries (such as Human Metabolome Database (HMDB, https://hmdb.ca), Mass Bank of North America (MoNA, https://mona.fiehnlab.ucdavis.edu/), and mzCloud (https://mzcloud.org)). The person of skill in the art would be able to identify lipids by, for example, annotating lipid MS/MS data using commercially available software tools, e.g. the Agilent Lipid Annotator software tool.

### Methods

The methods of the present invention comprise determining the levels of at least one biomarkers in an animal, wherein the at least 1 biomarker is selected from the biomarkers as listed in Table 1.

Suitably, the levels of at least 5, at least 6, at least 7, at least 8, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 50 or at least 75 biomarkers are determined, wherein at least 1 biomarker is selected from the biomarkers as listed in Table 1.

In some embodiments, the levels of at least 5, at least 10, at least 35 or at least 50 biomarkers are determined, wherein at least 1 biomarker is selected from the biomarkers as listed in Table 1.

Suitably, the levels of five or more biomarkers in an animal are determined, wherein at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70 or at least 80 biomarkers are selected from the biomarkers as listed in Table 1. Suitably, at least 2, at least 3, at least 4, at least 5, at least 10, at least 25, at least 35, at least 50 or at least 75 biomarkers are selected from the biomarkers as listed in Table 1.

In some embodiments, the levels of ten or more biomarkers in an animal are determined, wherein at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70 or at least 80 biomarkers are selected from the biomarkers as listed in Table 1. In some embodiments, at least 2, at least 3, at least 4, at least 5, at least 10, at least 25, at least 35, at least 50 or at least 75 biomarkers are selected from the biomarkers as listed in Table 1.

In some embodiments, the levels of fifteen or more biomarkers in an animal are determined, wherein at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70 or at least 80 biomarkers are selected from the biomarkers as listed in Table 1. In some embodiments, at least 2, at least 3, at least 4, at least 5, at least 10, at least 25, at least 35, at least 50 or at least 75 biomarkers are selected from the biomarkers as listed in Table 1.

In some embodiments, the levels of thirty-five or more biomarkers in an animal are determined, wherein at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70 or at least 80 biomarkers are selected from the biomarkers as listed in Table 1. In some embodiments, at least 2, at least 3, at least 4, at least 5, at least 10, at least 25, at least 35, at least 50 or at least 75 biomarkers are selected from the biomarkers as listed in Table 1.

In some embodiments, the levels of fifty or more biomarkers in an animal are determined, wherein at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70 or at least 80 biomarkers are selected from the biomarkers as listed in Table 1. In some embodiments, at least 2, at least 3, at least 4, at least 5, at least 10, at least 25, at least 50, or at least 75 biomarkers are selected from the biomarkers as listed in Table 1.

In some embodiments, the levels of five or more biomarkers are determined, wherein at least 1, at least 2, at least 3, at least 4, at least 5, at least 6, at least 7, at least 8, at least 9, at least 10, at least 15, at least 20, at least 25 or at least 30 biomarkers are selected from the biomarkers as listed in Table 2. In some embodiments, the method comprises determining the levels of at least the biomarkers listed in Table 2. In some embodiments, the method comprises determining the levels of the biomarkers listed in Table 2.

In some embodiments, the levels of five or more biomarkers are determined, wherein at least 1, at least 2, at least 3, at least 4, at least 5, at least 6, at least 7, at least 8, at least 9, at least 10, at least 11, at least 12, at least 13, at least 14, at least 15, at least 20 or at least 25 biomarkers are selected from the biomarkers listed as biomarkers 1 to 30 in Table 2. In some embodiments, the method comprises determining the levels of at least the biomarkers listed as biomarkers 1 to 30 in Table 2. In some embodiments, the method comprises determining the levels of the biomarkers listed as biomarkers 1 to 30 in Table 2. In some embodiments, the levels of five or more biomarkers are determined, wherein at least 1, at least 2, at least 3, at least 4, at least 5, at least 6, at least 7, at least 8, at least 9, at least 10, at least 11, at least 12, at least 13, at least 14, at least 15, at least 20, at least 25 or at least 30 biomarkers are selected from the biomarkers listed as biomarkers 1 to 35 in Table 2. In some embodiments, the method comprises determining the levels of at least the biomarkers listed as biomarkers 1 to 35 in Table 2. In some embodiments, the method comprises determining the levels of the biomarkers listed as biomarkers 1 to 35 in Table 2.

In some embodiments, the levels of at least 10 biomarkers are determined, wherein at least 10 of the biomarkers are selected from the biomarkers listed as biomarkers 1 to 30 in Table 2. In some embodiments, the at least 10 biomarkers comprise ACar 18:0, TG 16:0_17:1_18:1, TG 62:13, TG 54:8, TG 54:8, CE 18:0, PC 18:0_20:3, TG 17:0_18:1_18:2, PC 36:4 and N-Acetylleucine. In some embodiments, the at least 10 biomarkers comprise TG 16:0_18:1_18:2, TG 62:13, TG 58:9, DG 17:0_18:1, TG 18:1_18:2_22:0, TG 54:8, TG 20:4_20:4_20:5, TG 17:0_18:1_18:2, PC 36:4 and DG 16:0_18:1. In some embodiments, the at least 10 biomarkers comprise TG 15:0_18:1_18:2, TG 20:4_20:4_20:5, DG 17:0_18:1, TG 57:2, TG 17:0_18:1_18:2, L-Carnitine, DG 18:2_18:2, TG 16:0_17:0_18:1, DG 18:1_18:1 and TG 53:4. In some embodiments, the at least 10 biomarkers comprise PC 36:4, TG 62:13, DG 16:0_18:1, DG 18:1_18:2, TG 18:1_18:2_22:0, TG 17:0_18:1_18:1, TG 17:0_18:1_18:2, TG 15:0_18:1_18:2, TG 54:8, TG 57:2.

In some embodiments, the levels of at least 5 biomarkers are determined, wherein at least 5 of the biomarkers are selected from the biomarkers listed as biomarkers 1 to 30 in Table 2. In some embodiments, the at least 5 biomarkers comprise DG 18:2_18:2, DG 20:1_18:2, TG 54:8, CE 18:0, and DG 16:0_18:2. In some embodiments, the at least 5 biomarkers comprise TG 18:1_18:2_22:0, DG 18:1_18:1, L-Carnitine, TG 17:0_18:1_18:1, and TG 16:0_17:0_18:1. In some embodiments, the at least 5 biomarkers comprise DG 17:0_18:2, DG 18:1_18:2, DG 18:2_18:2, TG 18:1_18:2_22:0, and TG 54:8.

In some embodiments, the levels of five or more biomarkers are determined, wherein at least 1, at least 2, at least 3, at least 4, at least 5, at least 6, at least 7, at least 8, or at least 9 biomarkers are selected from the biomarkers listed as biomarkers 1 to 10 in Table 2. In some embodiments, the method comprises determining the levels of at least the biomarkers listed as biomarkers 1 to 10 in Table 2. In some embodiments, the method comprises determining the levels of the biomarkers listed as biomarkers 1 to 10 in Table 2.

In some embodiments, the levels of five or more biomarkers are determined, wherein at least 1, at least 2, at least 3 or at least 4 biomarkers are selected from the biomarkers listed as biomarkers 1 to 5 in Table 2. In some embodiments, the method comprises determining the levels of at least the biomarkers listed as biomarkers 1 to 5 in Table 2. In some embodiments, the method comprises determining the levels of the biomarkers listed as biomarkers 1 to 5 in Table 2.

In some embodiments, the levels of five or more biomarkers are determined, wherein at least 1, at least 2, at least 3, at least 4, at least 5, at least 6, at least 7, at least 8, at least 9, at least 10, at least 15, at least 20, at least 25, at least 30, at least 35, at least 40, at least 45 or at least 50 biomarkers are selected from the biomarkers as listed in Table 3. In some embodiments, the method comprises determining the levels of at least the biomarkers listed in Table 3. In some embodiments, the method comprises determining the levels of the biomarkers listed in Table 3.

In some embodiments, the levels of five or more biomarkers are determined, wherein at least 1, at least 2, at least 3, at least 4, at least 5, at least 6, at least 7, at least 8, at least 9, at least 10, at least 15, at least 20, at least 25, at least 30, at least 35, at least 40 or at least 45 biomarkers are selected from the biomarkers listed as biomarkers 1 to 50 in Table 3. In some embodiments, the method comprises determining the levels of at least the biomarkers listed as biomarkers 1 to 50 in Table 3. In some embodiments, the method comprises determining the levels of the biomarkers listed as biomarkers 1 to 50 in Table 3.

In some embodiments, the levels of five or more biomarkers are determined, wherein at least 1, at least 2, at least 3, at least 4, at least 5, at least 6, at least 7, at least 8, at least 9, at least 10, at least 11, at least 12, at least 13 or at least 14 biomarkers are selected from the biomarkers listed as biomarkers 1 to 15 in Table 3. In some embodiments, the method comprises determining the levels of at least the biomarkers listed as biomarkers 1 to 15 in Table 3. In some embodiments, the method comprises determining the levels of the biomarkers listed as biomarkers 1 to 15 in Table 3.

In some embodiments, the levels of five or more biomarkers are determined, wherein at least 1, at least 2, at least 3, at least 4, at least 5, at least 6, at least 7, at least 8 or at least 9 biomarkers are selected from the biomarkers listed as biomarkers 1 to 10 in Table 3. In some embodiments, the method comprises determining the levels of at least the biomarkers listed as biomarkers 1 to 10 in Table 3. In some embodiments, the method comprises determining the levels of the biomarkers listed as biomarkers 1 to 10 in Table 3.

In some embodiments, the levels of at least 10 biomarkers are determined, wherein at least 10 of the biomarkers are selected from the biomarkers listed as biomarkers 1 to 30 in Table 3. In some embodiments, the at least 10 biomarkers comprise TG 18:0_18:1_18:1, DG 17:0_18:2, TG 55:2, TG 50:1, DG 18:0, DG 18:1_22:5, LPC 16:0/0:0, DG 17:1_18:2, DG 16:1_18:2 and TG 18:2_20:4_21:0. In some embodiments, the at least 10 biomarkers comprise DG 18:1_22:5, TG 52:5, DG 18:1_20:4, TG 51:1, TG 48:6_254, TG 56:7, N-Acetylputrescine, DG 18:0_22:0, TG 16:0_18:2_18:2 and TG 18:0_18:1_18:1. In some embodiments, the at least 10 biomarkers comprise HexCer_NS d18:1_16:0, TG 55:2, DG 16:0_18:1, DG 18:1_20:4, 1-Phenylethanol, 10-Hydroxydecanoate, TG 58:11, TG 52:5, TG 18:0_18:1_18:1 and TG 50:4. In some embodiments, the at least 10 biomarkers comprise TG 58:11, TG 52:6, LPC 16:0/0:0, TG 52:5, DG 17:0_18:2, TG 56:6, TG 52:8, TG 18:2_20:4_21:0, 10-Hydroxydecanoate and TG 50:1. In some embodiments, the at least 10 biomarkers comprise TG 50:4, TG 55:8, 1-Phenylethanol, TG 50:1, TG 18:0_18:1_18:1, N-Acetylputrescine, DG 16:0_18:1, TG 52:6, TG 56:7 and 10-Hydroxydecanoate. In some embodiments, the at least 10 biomarkers comprise TG 55:8, HexCer_NS d18:1_16:0, TG 52:6, TG 18:0_18:1_18:1, DG 17:1_18:2, TG 48:6, TG 50:1, N-Acetylputrescine, TG 50:4 and TG 16:0_18:2_18:2.

In some embodiments, the levels of at least 5 biomarkers are determined, wherein at least 5 of the biomarkers are selected from the biomarkers listed as biomarkers 1 to 30 in Table 3. In some embodiments, the at least 5 biomarkers comprise TG 52:8, DG 16:1_18:2, TG 52:6, TG 56:7 and DG 18:1_20:4. In some embodiments, the at least 5 biomarkers comprise TG 56:6, DG 16:0_18:1, 10-Hydroxydecanoate, HexCer_NS d18:1_16:0 and TG 52:5. In some embodiments, the at least 5 biomarkers comprise LPC 16:0/0:0, DG 18:0_22:0, TG 52:8, TG 58:11 and TG 18:2_20:4_21:0. In some embodiments, the at least 5 biomarkers comprise 1-Phenylethanol, HexCer_NS d18:1_16:0, TG 50:1, DG 16:1_18:2 and DG 18:0_22:0. In some embodiments, the at least 5 biomarkers comprise TG 50:4, DG 16:0_18:1, TG 55:2, TG 56:7 and DG 18:0_20:4. In some embodiments, the at least 5 biomarkers comprise TG 48:6, TG 18:0_18:1_18:1, DG 18:1_20:4, TG 16:0_18:2_18:2 and TG 52:6.

In some embodiments, the levels of at least 5 biomarkers are determined, wherein at least 5 of the biomarkers are selected from the biomarkers listed as biomarkers 1 to 10 in Table 3. In some embodiments, the at least 5 biomarkers comprise DG 16:0_18:2, 1-Phenylethanol, DG 16:0_18:1, TG 18:2_20:4_21:0 and TG 55:8. In some embodiments, the at least 5 biomarkers comprise DG 16:1_18:2, 10-Hydroxydecanoate, DG 16:0_18:2, DG 18:0_20:4 and TG 18:2_20:4_21:0. In some embodiments, the at least 5 biomarkers comprise TG 55:2, TG 55:8, TG 18:2_20:4_21:0, 1-Phenylethanol and 10-Hydroxydecanoate. In some embodiments, the at least 5 biomarkers comprise 1-Phenylethanol, TG 55:8, TG 18:2_20:4_21:0, DG 16:1_18:2 and DG 16:0_18:2. In some embodiments, the at least 5 biomarkers comprise TG 55:8, DG 16:1_18:2, DG 18:1_22:5, DG 16:0_18:1 and DG 18:0_20:4. In some embodiments, the at least 5 biomarkers comprise DG 16:0_18:1, DG 18:1_22:5, 10-Hydroxydecanoate, TG 55:8 and TG 55:2.

In some embodiments, the levels of five or more biomarkers are determined, wherein at least 1, at least 2, at least 3 or at least 4 biomarkers are selected from the biomarkers listed as biomarkers 1 to 5 in Table 3. In some embodiments, the method comprises determining the levels of at least the biomarkers listed as biomarkers 1 to 5 in Table 3. In some embodiments, the method comprises determining the levels of the biomarkers listed as biomarkers 1 to 5 in Table 3.

### Further Biomarkers

In some embodiments, the methods of the present invention may comprise determining the levels of additional biomarkers in an animal that are not listed in Tables 1-3. The methods of the present invention may comprise determining the levels of additional biomarkers of any type. In some embodiments, the methods of the present invention may comprise determining the levels of additional biomarkers that are not metabolites in an animal, further to at least 1 metabolite biomarker selected from the biomarkers as listed in Table 1.

Suitably, the additional biomarkers may be any type of biological molecule or physiological characteristic. Biomarkers according to the present invention may include, for example, metabolites, further small molecules not limited to metabolites, proteins, mRNA, genes (e.g. methylation), cells, and ions.

### Determination of Biomarkers Indicative of Biological Age of an Animal

The present invention comprises determining the levels of biomarkers in an animal and determining the biological age of an animal from said levels of biomarkers.

The provision of levels of biomarkers that are indicative of biological age may be achieved through training datasets and machine learning approaches, for example. Suitably, the machine learning approaches may be supervised machine learning approaches. Suitably, the model may be a multinomial regression, support-vector machine (SVM), a random forest or an Adaptive Boosting (AdaBoost) model.

By way of example, levels of biomarkers in individual animals may be trained against a dataset comprising animals of known chronological age. Suitably, levels of biomarkers may be trained against a dataset comprising animals that were known to subsequently develop age-related disease or die after the samples from which the dataset was generated were taken.

Suitably, the chronological age of an animal may be referred to as the age of an animal.

For example, models of biomarkers indicative of biological age may be provided by training a dataset of a range of biomarker levels in individuals against a training dataset of animals with a known age using a machine learning framework, and testing against a with-held cohort to validate the veracity of the model.

The machine learning framework may comprise fitting a multinomial model, a random forest, an AdaBoost, a SVM (support vector machine), penalized multinomial logistic regression or other model used to predict multi class outcomes.

Suitably, the machine learning framework may be used to determine a model comprising a set of biomarkers and biomarker levels that are indicative of biological age.

The model may comprise the levels of biomarkers; wherein the level of a biomarker is considered in the model by multiplying by a coefficient value.

The coefficient value for each parameter typically depends on the measurement units of all the variables in the model. As would be understood by the skilled person, the value for each coefficient value will therefore depend on, for example, the number and nature of the different parameters used in the model and the nature of the training data provided. Accordingly, routine statistical methods may be applied to a training data set in order to arrive at coefficient values.

Suitably, the machine learning platform may comprise one or more deep neural networks. Neural Networks are collections of neurons (also called units) connected in an acyclic graph. Neural Network models are often organized into distinct layers of neurons. For most neural networks, the most common layer type is the fully-connected layer in which neurons between two adjacent layers are fully pairwise connected, but neurons within a single layer share no connections. One of the main features of deep neural networks is that neurons are controlled by non-linear activation functions. This non-linearity combined with the deep architecture make possible more complex combinations of the input features leading ultimately to a wider understanding of the relationships between them and as a result to a more reliable final output. Deep neural networks have been applied for many types of data ranging from structural data to chemical descriptors or transcriptomics data.

Suitably, the machine learning platform comprises one or generative adversarial networks. Suitably, the machine learning platform comprises an adversarial autoencoder architecture. Suitably, the machine learning platform comprises a feature importance analysis for ranking biomarkers by their importance in determining biological age.

### Comparison to a Reference or Control

The present method may comprise a step of comparing the difference in the level(s) of one or more biomarker in the test sample to one or more reference or controls. The level(s) of one or more biomarker in the reference or control may be associated with biological age. In some embodiments, the reference value is a value obtained previously for a subject or group of subjects with a known biological age. The reference value may be based on a known level(s) of one or more biomarker, e.g. a mean or median level, from a group of subjects with known biological age.

The reference level(s) of one or more biomarker may comprise level(s) of one or more biomarker from at least 1, at least 2, at least 4, at least 10, at least 20, at least 40, at least 80, at least 100, at least 150, at least 200, at least 300, at least 400 animals, at least 500, at least 750 or at least 1000 animals.

### Age-Associated Diseases

Biological age is a risk factor for a variety of diseases. In particular, a biological age greater than the chronological age of an animal may be predictive of a greater risk of disease. A biological age equal to the chronological age of an animal may also be predictive of a greater risk of disease

As an example, dogs with greater biological ages are more likely to suffer from renal disease, immune system or immune-related diseases or disorders, cancer, cardiovascular diseases, neurodegenerative diseases and musculoskeletal diseases.

Suitably, the methods of the present invention may comprise determining the risk of an age-related disease. Suitably, the risk of an age-related disease may be determined from the determined biological age of an animal. The risk of an age-related disease may be determined from a determined numerical biological age, and/or from a determined categorical biological age. Suitably, the risk of an age-related disease may be determined from the comparison of the determined biological age of an animal with the chronological age of an animal. For example, where an animal is determined to have a biological age equal to or greater than its chronological age, the risk of an age-related disease may be increased. Where an animal is determined to have a biological age younger than its chronological age, the risk of an age-related disease may be reduced. Suitably, the determination of the risk of an age-related disease may comprise the determination of the biological age of an animal according to the present invention, combined with other suitable health indicators known in the art. For example, the determined biological age of an animal may be combined with other disease indicators or health metrics known in the art, such as an animal's weight, blood glucose, cholesterol or triglyceride levels, inflammation markers, oxidative stress markers, or results of a physical examination.

It is to be appreciated that all references herein to treatment include curative, palliative and prophylactic treatment; although in the context of the invention references to preventing are more commonly associated with prophylactic treatment. Treatment may also include arresting progression in the severity of a disease.

### Method for Selecting a Dietary, Pharmacological, or Lifestyle Regime

In a further aspect, the present invention provides a method for selecting a dietary, pharmacological, or lifestyle regime for a subject. Suitably, the method is for selecting a dietary, pharmaceutical, or lifestyle regimen for a dog.

Suitably, the method for selecting a dietary, pharmacological, or lifestyle regime for a subject comprises determining the biological age of the subject by methods according to the present invention. A dietary, pharmacological, or lifestyle regimen may be selected based on the determined biological age of the subject. In some embodiments, the selection of a dietary, pharmacological, or lifestyle regime for a subject is based on a determined numerical biological age of a subject. In some embodiments, the selection of a dietary, pharmacological, or lifestyle regime for a subject is based on a determined categorical biological age of a subject. In some embodiments, the selection of a dietary, pharmacological, or lifestyle regime for a subject is based on a comparison between the determined biological age of a subject and the subject's chronological age. Suitably, a dietary, pharmacological, or lifestyle regime may be selected for a subject wherein the determined biological age is equal to or greater than the subject's chronological age.

The dietary, pharmacological, or lifestyle regime may be applied to the dog for any suitable period of time. By way of example, the dietary, pharmacological, or lifestyle regime may be applied for at least 2, at least 4, at least 8, at least 16, at least 32, or at least 64 weeks. The dietary, pharmacological, or lifestyle regime may be applied for at least 3, at least 6, at least 12, at least 24, at least 36, at least 48 or at least 60 months. Suitably, the dietary, pharmacological, or lifestyle regime may be applied for at least 1, at least 2, at least 3, at least 4, at least 5, at least 6, at least 7, at least 8, at least 9 or at least 10 years. Suitably, the dietary, pharmacological, or lifestyle regime may be applied for the lifetime of the subject.

Suitably, the modification is a dietary intervention as described herein. By the term “dietary intervention” it is meant an external factor applied to a subject which causes a change in the subject's diet. More preferably the dietary intervention includes the administration of at least a dietary product or a dietary regimen or a nutritional or nutraceutical supplement.

The dietary regime may be a meal, a regime of meals, a supplement or a regime of supplements, or combinations of a meal and a supplement, or combinations of a meal and multiple supplements.

The dietary intervention or dietary product described herein may be any suitable dietary regime, for example, a calorie-restricted diet, a senior diet, a low protein diet for young animals or a high protein diet for senior animals, an anti-inflammatory diet, potassium supplement diet, polyunsaturated fatty acids (PUFA) supplement diet, anti-oxidant supplement diet, a vitamin B supplement diet, liquid diet, selenium supplement diet, omega 3-6 ratio diet, or diets supplemented with carnitine, branched chain amino acids or derivatives, nucleotides, nicotinamide precursors such as nicotinamide mononucleotide (MNM) or nicotinamide riboside (NR) or any combination of the above.

Suitably, the dietary intervention or dietary product may be a calorie-restricted diet, a high-calorie diet, a senior diet, or a low protein diet for young animals and a high protein for senior animals. Suitably, the dietary intervention or dietary product may be a calorie-restricted diet. Suitably, the dietary intervention or dietary product may be a low protein diet for young animals and a high protein diet for senior animals.

A dietary intervention may be determined based on the baseline maintenance energy requirement (MER) of the subject. Suitably, the MER may be the amount of food that stabilizes the dog's body weight (less than 5% change over three weeks). Suitably the MER may be predicted by the determined biological age of an animal.

By way of example, it is generally understood that younger, growing dogs benefit from a high energy/high protein diet; however, older dogs may have a lower energy requirement and therefore diets can be appropriately modified. In particular, many manufacturers produce a ‘senior’ range of dog food which is lower in calories, higher in fibre and protein and suitable levels of fat for an older dog.

Suitably, a calorie-restricted diet may comprise about 50%, about 55%, about 60%, about 65%, about 75%, about 80%, about 85%, or about 90% of the animal's MER. Suitably, a calorie-restricted diet may comprise about 60% or about 75% of the animal's MER.

Suitably, a low-protein diet may comprise less than 20% protein (% dry matter). For example, a low-protein diet may comprise less than 19% (% dry matter). A high protein diet may comprise more than 20% protein (% dry matter), for example a high protein diet may comprise more than 30% protein (% dry matter).

The dietary intervention may comprise a food, supplement and/or drink that comprises a nutrient and/or bioactive that mimics the benefits of caloric restriction (CR) without limiting daily caloric intake. For example, the food, supplement and/or drink may comprise a functional ingredient(s) having CR-like benefits. Suitably, the food, supplement and/or drink may comprise an autophagy inducer. Suitably, the food, supplement and/or drink may comprise fruit and/or nuts (or extracts thereof). Suitable examples include, but are not limited to, pomegranate, strawberries, blackberries, camu-camu, walnuts, chestnuts, pistachios, pecans. Suitably, the food, supplement and/or drink may comprise probiotics with or without fruit extracts or nut extracts.

By way of example, animals with a low biological age may be more active than animals with a high biological age. In some embodiments, the animal subject is a dog. Suitably, a low biological age may be a biological age of 2-6 years (i.e. a “young” dog as categorised in categorical biological age). A dog food composition having a ratio of energy from protein to energy from fat below 0.80 may be advantageous to dogs with a low biological age. A food composition high in protein and high in fat is particularly well adapted for dogs with a low biological age. Typically, a dog food composition for dogs with a low biological age has from about 20-30% protein and from about 15-25% fat. Indeed, a food composition dense in energy from fat will provide a dog with a low biological age with sufficient energy for the moderate to very intense activities (i.e., brisk walk to fast run) in which it spontaneously gets involved. Furthermore, the energy from protein to the energy from fat ratio is found to be advantageous in such a food composition for maintaining the lean body mass of dogs with a low biological age.

Similarly, animals with a high biological age may be less active than animals with a low biological age. In some embodiments, the animal subject is a dog. Suitably, a high biological age may be a biological age of 7+ years (i.e. a “senior” dog as categorised in categorical biological age). A particularly well adapted dog food composition for a dog with a high biological age may have the ratio of energy from protein to energy from fat in such a food composition greater than 0.80. More specifically, a protein content from about 20-30% or higher and less than about 15% fat. Because they may have a low resting metabolic rate, such a food composition is ideally adapted to dogs with a high biological age. The composition will have the effect of limiting the fat and/or carbohydrate intake of dogs with a high biological age and therefore their tendency to be overweight.

The modification in lifestyle may be any change as described herein, e.g. a change in exercise regime.

Similar to a dietary intervention, the determination of a biological age may allow a determination to switch the test subject to an appropriate exercise regime.

Ideal activity level and type may differ according to determined biological age. For example, wherein the animal subject is a dog, a dog with a high biological age may be spontaneously engaged in mild (e.g., slow walking), moderate (e.g., brisk walking) or occasionally intense (e.g., running) activity types. A dog with a low biological age, in comparison, may mainly be voluntarily involved in moderate, intense or very intense (e.g., fast running) activities.

The pharmacological regime may refer to administration of a therapeutic modality or regimen. The modality may be a modality useful in treating and/or preventing—for example—arthritis, dental diseases, endocrine disorders, heart disease, diabetes, liver disease, kidney disease, prostate disorders, cancer, immune-related diseases or disorders, and behavioural or cognitive disorders.

Suitably, a therapeutic modality or regimen may include any suitable therapeutic agent. A therapeutic modality or regimen may include any therapy or therapeutic agent suitable to treat, prevent, diagnose or reduce the risk of a disease or condition. In some embodiments, a therapeutic modality or regimen may be suitable for improving the biological age of a subject. Suitably, a therapeutic modality or regimen may include any of small molecules, biologics, and gene therapies.

Suitably, prophylactic therapies may be administered to a subject identified as being at risk of such disorders due to the biological age of the subject. In other embodiments, subjects determined to be at risk of certain conditions due to biological age may be monitored more regularly so that diagnosis and treatment can begin as early as possible.

### Method for Determining the Efficacy of a Dietary, Pharmacological or Lifestyle Regime

In a further aspect, the present invention provides a method for determining the efficacy of a dietary, pharmacological, or lifestyle regime that is administered to an animal subject. Suitably, the present invention provides a method for determining the efficacy of a dietary, pharmaceutical, or lifestyle regime in improving the biological age of an animal subject. Suitably, the method is for determining the efficacy of a dietary, pharmaceutical, or lifestyle regime in improving the biological age of an animal subject wherein the biological age of the subject has been determined by the methods disclosed herein.

Suitably, a method for determining the efficacy of a dietary, pharmacological, or lifestyle regime administered to an animal subject comprises determining the biological age of the subject according to the methods of the present invention prior to the administration of the dietary, pharmacological, or lifestyle regime to the subject, determining the biological age of the subject according to the methods of the present invention after the administration of the dietary, pharmacological, or lifestyle regime to the subject, and determining if there has been a change in the biological age of the subject between prior to the administration and after the administration of the dietary, pharmacological, or lifestyle regime to the subject.

Suitably, at least one of the determination of the subject's biological age prior to the administration of the dietary, pharmacological, or lifestyle regime to the subject, or the determination of the subject's biological age after the administration of the dietary, pharmacological, or lifestyle regime to the subject may be achieved by a method of determining the biological age of a subject according to the present invention. Alternative methods of determining biological age of a subject may be used instead of, or in addition to, the methods according to the present invention, and further determinations of biological age of the subject prior to or after administration of the dietary, pharmacological, or lifestyle regime to the subject may be carried out, so long as at least one of the determinations of the biological age of the subject is carried out according to the methods of the present invention.

In some embodiments, the biological age of the subject may be determined multiple times prior to the administration of the dietary, pharmacological, or lifestyle regime to the subject. In some embodiments, the biological age of the subject may be determined multiple times after the administration of the dietary, pharmacological, or lifestyle regime to the subject.

Suitably, the determination of the biological age of the subject after administration of the dietary, pharmacological, or lifestyle regime to the subject may be carried out a suitable period after the administration of the dietary, pharmacological, or lifestyle regime to the subject. The period may be, for instance, at least 1 day, at least 2 days, at least 3 days, at least 4 days, at least 5 days, at least 6 days, at least 7 days, at least 14 days, at least 3 weeks, at least 4 weeks, at least 5 weeks, at least 6 weeks, at least 7 weeks, at least 8 weeks, at least 3 months, at least 4 months, at least 5 months, at least 6 months, or at least a year after administration of the dietary, pharmacological, or lifestyle regime to the subject.

In some embodiments, the method may comprise the selection of an alternative dietary, pharmacological, or lifestyle regime for administration to an animal subject, where the previous dietary, pharmacological, or lifestyle regime is determined to be inefficient in improving the biological age of the subject. In some embodiments, the method may comprise changing the administration frequency or dosage of a pharmacological regime that is determined to be inefficient in improving the biological age of the subject. In some embodiments, the method may comprise adjusting the dietary regime administered to the animal subject, wherein the dietary regime is determined to be inefficient in improving the biological age of the subject. Suitably the dietary regime may be adjusted to change the amount of food provided to the subject, the feeding time, and/or the frequency of feeding. In some embodiments, the method may comprise adjusting the lifestyle regime administered to the subject, wherein the lifestyle regime is determined to be inefficient in improving the biological age of the subject. Suitably, the lifestyle regime may be adjusted to change the amount, frequency and/or time of exercise of the subject.

### Method for Determining the Likelihood of Benefit of a Dietary, Pharmacological or Lifestyle Regime

In a further aspect, the present invention provides a method for determining the likelihood that an animal subject will benefit from a dietary, pharmacological, or lifestyle regime. Suitably, the dietary, pharmacological, or lifestyle regime is for improving the biological age of the animal subject.

Suitably, a method for determining the likelihood that an animal subject will benefit from a dietary, pharmacological, or lifestyle regime for improving the biological age of a subject comprises determining the biological age of the subject by a method described herein, and identifying the subject as being likely to benefit from a dietary, pharmacological, or lifestyle regime for improving the biological age of a subject based on the determined biological age. Suitably, a subject may be likely to benefit from a dietary, pharmaceutical, or lifestyle regime for improving biological age of a subject where the subject has a determined biological age that is higher than the chronological age of the subject. In some embodiments, a subject may be likely to benefit from a dietary, pharmaceutical, or lifestyle regime for improving biological age of a subject wherein the subject has a determined biological age that is high. Where the subject is a dog, for instance, a high biological age may be a determined biological age of at least 7 years, at least 8 years, at least 9, at least 10, at least 11, at least 12, at least 13, at least 14, or at least 15 years. Suitably, where the subject is a dog, a high biological age is a determined biological age of at least 7 years.

In some embodiments, a subject may be likely to benefit from a dietary, pharmaceutical, or lifestyle regime for improving biological age of a subject where the subject has a determined biological age that equal to or close to the chronological age of the subject. Suitably, where the biological age is determined in years, close to may refer to where the determined biological age is within 2 years, within 1 year, or within half a year of the chronological age of the subject. Suitably, where the biological age is determined in months, close to may refer to where the determined biological age is within 24 months, within 18 months, within 12 months, within 9 months, within 6 months, within 4 months, within 3 months, within 2 months or within 1 month of the chronological age of the subject. Suitably, where the biological age is determined in years, equal to may refer to where the determined biological age is the same age in years as the chronological age of the subject. Suitably, where the biological age is determined in months, equal to may refer to where the determined biological age is the same age in months as the chronological age of the subject.

The present invention may thus advantageously enable the identification of dogs that are expected to respond particularly well to a given intervention (e.g. dietary, pharmacological or lifestyle regime). The intervention can thus be applied in a more targeted manner to dogs that are expected to respond due to their determined biological age.

### Use of a Dietary Intervention or Pharmaceutical Product

In one aspect, the present invention provides a dietary or pharmacological product for use in improving the biological age of an animal subject, wherein the dietary or pharmaceutical product is administered to the subject. Suitably, the dietary or pharmaceutical product is administered to a subject for which a biological age has been determined by the methods described herein.

Suitably, an improvement in the biological age of an animal subject may be a reduction of the biological age of the subject. In some embodiments, an improvement in the biological age of an animal subject may be a reduction of the biological age of the subject relative to the chronological age of the subject. For instance, an improvement in the biological age of an animal subject may be a reduction of the biological age of the subject to match or be more similar to the chronological age of the subject. In some embodiments, an improvement in the biological age of an animal subject may be a reduction of the biological age of the subject to be lower than the chronological age of the subject.

In one aspect, the present invention provides a dietary or pharmacological product for use in reducing the risk of an age-related disease in an animal subject, wherein the dietary or pharmaceutical product is administered to the subject. Suitably, the dietary or pharmaceutical product is administered to a subject for which a biological age has been determined by the methods described herein. In one aspect, the dietary or pharmacological product is administered to an animal subject which has been determined to be at risk of an age-related disease by determination of the subject's biological age by the methods described herein.

Suitably, where reference is made in the present application to a dietary, pharmaceutical, or lifestyle regime, it is to be understood that this encompasses supplements.

### Ecosystem

Suitably, the present methods may comprise an ‘ecosystem’; in particular a digital ecosystem. Suitably, the present methods may comprise providing a sample obtained from the animal subject, optionally using a kit according to present invention; and (b) providing the sample (e.g. by mailing) for subsequent determination of the level of one or more biomarker(s) in the sample.

The level of one or more biomarker(s) may then be used according to any of the present methods; preferably using a computer system or a computer program product according to the present invention.

The computer system or computer program may then prepare and share a report detailing the outcome of analysis/method in the form of e.g. selecting or recommending a suitable lifestyle regime, dietary regime or therapeutic intervention for a dog or any other outcome of the present methods.

### Computer-Readable Medium and Computer System

The present methods may be performed using a computer. Accordingly, the present methods may be performed in silico.

Suitably, the computer may prepare and share a report detailing the outcome of the present methods.

The methods described herein may be implemented as a computer program running on general purpose hardware, such as one or more computer processors. In some embodiments, the functionality described herein may be implemented by a device such as a smartphone, a tablet terminal or a personal computer.

In one aspect, the present invention provides a computer program product comprising computer implementable instructions for causing a programmable computer to determine the biological age of an animal as described herein. Suitably, the computer program product comprises a computer-readable medium comprising said computer-implementable instructions.

In another aspect, the present invention provides a computer program product comprising computer implementable instructions for causing a device to determine the biological age of an animal subject; and optionally select a suitable dietary, pharmacological, or lifestyle regime for the subject based on the subject's biological age, as determined by the present method. The computer program product may also be given additional parameters or characteristics for the subject. Suitably, the computer program product comprises a computer-readable medium comprising said computer-implementable instructions.

In another aspect, the present invention provides a computer program product comprising computer implementable instructions for causing a device to determine whether an animal subject is at a risk of developing an age-related disease based on the subject's biological age; and optionally select a suitable dietary, pharmacological, or lifestyle regime for the subject based on the subject's risk of developing an age-related disease based on the subject's biological age, as determined by the present method. The computer program product may also be given additional parameters or characteristics for the subject. Suitably, the computer program product comprises a computer-readable medium comprising said computer-implementable instructions.

In one aspect, the present invention provides a computer system for determining the biological age of an animal as described herein. Suitably, the computer program product comprises a computer-readable medium comprising said computer-implementable instructions.

In another aspect, the present invention provides a computer system for determining the biological age of an animal subject; and optionally select a suitable dietary, pharmacological, or lifestyle regime for the subject based on the subject's biological age, as determined by the present method.

In another aspect, the present invention provides a computer system for determining whether an animal subject is at a risk of developing an age-related disease based on the subject's biological age; and optionally select a suitable dietary, pharmacological, or lifestyle regime for the subject based on the subject's risk of developing an age-related disease based on the subject's biological age, as determined by the present method.

In one embodiment, the user inputs into the computer system levels in an animal subject of five or biomarkers as defined herein. The computer system then processes this information and provides a determination of biological age. Alternatively, the computer system then processes this information and provides a determination of a suitable dietary, pharmacological, or lifestyle regime for the dog based on the biological age, as determined by the present method.

The computer system or device may generally be a server on a network. However, any computer system or device may be used as long as it can process biomarker data and/or additional parameters or characteristic data using a processor, a central processing unit (CPU) or the like. The computer system or device may, for example, be a smartphone, a tablet terminal or a personal computer and output information indicating the determined biological age of the subject or a determination of a suitable lifestyle or dietary regime for the subject based on the determined biological age.

Various preferred features and embodiments of the present invention will now be described by way of non-limiting examples. This disclosure is not limited by the exemplary methods and materials disclosed herein, and any methods and materials similar or equivalent to those described herein can be used in the practice or testing of embodiments of this disclosure. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes”, “containing”, or “contains”, and are inclusive or open-ended and do not exclude additional, non-recited members, elements or steps. The terms “comprising”, “comprises” and “comprised of” also include the term “consisting of”.

Numeric ranges are inclusive of the numbers defining the range. As used herein the term “about” means approximately, in the region of, roughly, or around. When the term “about” is used in conjunction with a numerical value or range, it modifies that value or range by extending the boundaries above and below the numerical value(s) set forth. In general, the terms “about” and “approximately” are used herein to modify a numerical value(s) above and below the stated value(s) by 10%.

The publications discussed herein are provided solely for their disclosure prior to the filing date of the present application. Nothing herein is to be construed as an admission that such publications constitute prior art to the claims appended hereto.

All publications mentioned in the specification are herein incorporated by reference.

### EXAMPLES

The invention will now be further described by way of examples, which are meant to serve to assist one of ordinary skill in the art in carrying out the invention and are not intended in any way to limit the scope of the invention.

### Illustrative Method for Determining Biological Age in Dogs Using Metabolomics

### Dataset Description

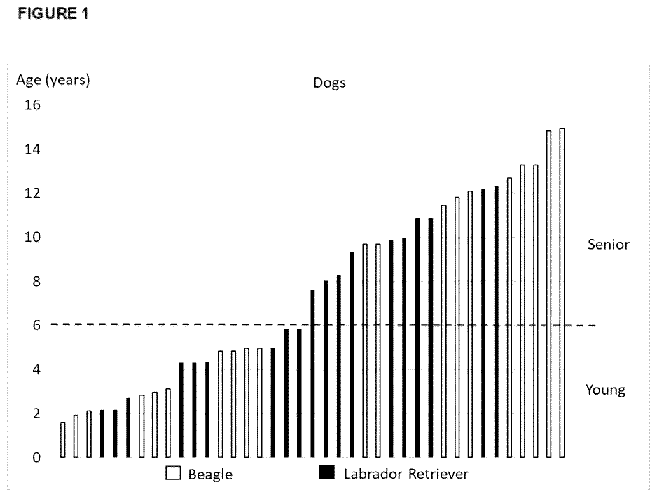

A biological age determination tool using metabolomic data was developed using a pet cohort composed of 39 dogs. The cohort was split into two groups. The young group was based on 2-6 year-olds. The senior group was based on 7+ year olds (FIG. 1).

Dogs were fed a complete and balanced diet at 100% of their estimated MERs for 5 weeks to stabilize their body weight (less than 5% changes in three weeks) by adjusting their food intake, and to standardize their metabolomic profiles. At the end of 5 weeks, a fasted blood sample was collected. Serum samples were aliquoted into two tubes with 250 μl per tube and two tubes with 125 μl per tube.

39 dog serum samples were used in the study.

### Metabolomic Analysis

### Sample Preparation

Subject serum, which had been stored at −80° C. upon collection, was thawed on ice. A 50 μL aliquot was transferred onto the solid-phase-extraction (SPE)-system CAPTIVA-EMR Lipid 96-wellplate (Agilent Technologies) before addition of 250 μL of acetonitrile containing 1% formic acid (v/v), or alternatively methanol:ethanol 1:1 (v/v), and 10 UM internal standard (consisting of uniformly ¹³C and ¹⁵N labeled amino acids from Cambridge Isotope Laboratories, Inc.). The samples were mixed for 1 min at 360 rpm on an orbital shaker at room temperature prior to a 10 min incubation period at 4° C. Afterwards, 200 μL 80% acetonitrile in water (v/v) were added to the samples. The samples were mixed on an orbital shaker (360 rpm) for an additional 10 min at room temperature to complete sample mixing. The samples were then eluted into a 96-deepwell collection plate by centrifugation (10 min, 57 g, 4° C. followed by 2 min, 1000 g, 4° C.). Polar eluates were stored at −80° C. until the day of analysis, on which thawed eluates were subjected to liquid chromatography/mass spectrometry (LC/MS) analysis.

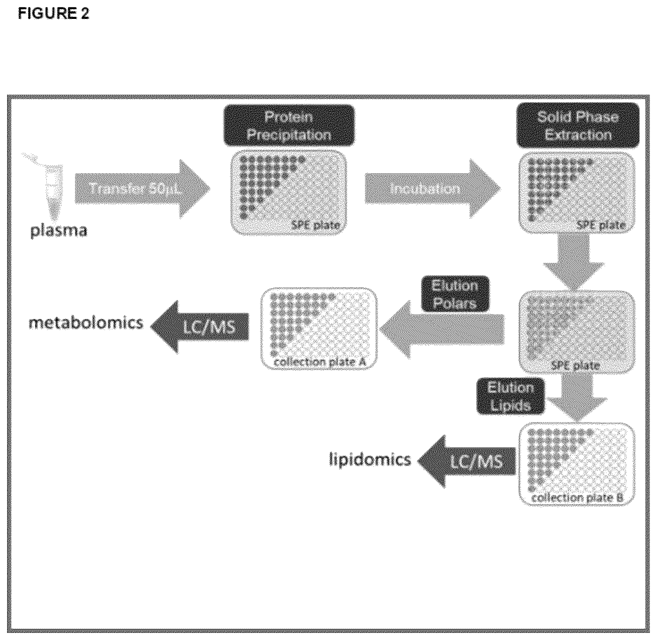

The SPE plates were then washed twice with 500 μL 80% acetonitrile in water (v/v). Lipids still bound to the SPE-material were then released into a second elution plate, in two elution steps, applying 2×500 μL 1:1 methyl-tert-butyl ether:methanol (v/v) onto the SPE cartridge and centrifuging for 2 min at 1000 g and 4° C. The combined eluates were dried under a stream of nitrogen (Biotage SPE Dry Evaporation System) at room temperature and reconstituted with 100 μL 1:1 2-propanol:methanol (v/v) prior to LC/MS analysis. A schematic of this workflow is shown in FIG. 2.

### LC/MS Analysis of Polar Metabolites

An aliquot of 2 μL of polar metabolite extract was subjected to LC/MS analysis by using an Agilent 1290 Infinity II liquid-chromatography (LC) system coupled to an Agilent 6540 Quadrupole-Time-of-Flight (Q-TOF) mass spectrometer with a dual Agilent Jet Stream electrospray ionization source. Metabolites were separated on a SeQuant® ZIC®-pHILIC column (100×2.1 mm, 5 μm, polymer, Merck-Millipore) including a ZIC®-pHILIC guard column (2.1 mm×20 mm, 5 μm). The column compartment temperature was maintained at 40° C. and the flow rate was set to 250 μL·min-1. The mobile phases consisted of A: 95% water, 5% acetonitrile, 20 mM ammonium bicarbonate, 0.1% ammonium hydroxide solution (25% ammonia in water), 2.5 μM medronic acid, and B: 95% acetonitrile, 5% water, 2.5 μM medronic acid.

The following linear gradient was applied: 0 to 1 min, 90% B; 1 to 12 min, 35% B; 12 to 12.5 min, 25% B; 12.5 to 14.5 min, 25% B; 14.5 to 15 min, 90% B followed by a re-equilibration phase of 4 min at 400 μL·min-1 and 2 min at 250 μL·min-1. Metabolites were detected in positive and negative ion mode with the following source parameters: gas temperature 200° C., drying gas flow 10 L·min-1, nebulizer pressure 44 psi, sheath gas temperature 300° C., sheath gas flow 12 L·min-1, VCap 3000 V, nozzle voltage 2000 V, Fragmentor 100 V, Skimmer 65 V and October 1 RF Vpp 750 V, m/z range 50-1700. Data were acquired under continuous reference mass correction at m/z 121.0509 and 922.0890 (positive ion mode), m/z 119.036 and 966.0007 (negative ion mode). Samples were randomized before analysis. In addition, a quality-control sample was injected after every 12th sample to monitor signal stability of the instrument. Iterative MS/MS fragmentation data were acquired on a pooled plasma sample utilizing the MassHunter Acquisition Software (Agilent Technologies).

### LC/MS Analysis of Lipid Metabolites

An aliquot of 2 μL of lipid extract was subjected to LC/MS analysis by using an Agilent 1290 Infinity II LC-system coupled to an Agilent 6545 Q-TOF mass spectrometer with a dual Agilent Jet Stream electrospray ionization source. Lipids were separated on an Acquity UPLC® HSS T3 column (2.1×150 mm, 1.8 μm) including an Acquity UPLC® HSS T3 VanGuard Pre-Column (2.1×5 mm, 1.8 μm) at a temperature of 60° C., and a flow rate of 250 L·min-1. The mobile phases consisted of A: 60% acetonitrile, 40% water, 0.1% formic acid, 10 mM ammonium formate, 2.5 μM medronic acid, and B: 90% 2-propanol, 10% acetonitrile, 0.1% formic acid, 10 mM ammonium formate (dissolved in 1 mL water). The following linear gradient was used: 0-2 min, 30% B; 2-17 min, 75% B; 17-20 min, 85%; 20-23 min, 100% B; 23-26 min, 100% B; 26-27 min, 30% B followed by a re-equilibration phase of 4 min.

Lipids were detected in positive and negative ion mode with the following source parameters: gas temperature 250° C., drying gas flow 11 L·min-1, nebulizer pressure 35 psi, sheath gas temperature 300° C., sheath gas flow 12 L·min-1, VCap 3000 V, nozzle voltage 500 V, Fragmentor 160 V, Skimmer 65 V and October 1 RF Vpp 750 V, m/z range 50-1700. Data were acquired under continuous reference mass correction at m/z 121.0509 and 922.0890 in positive ion mode, and m/z 119.036 and 966.0007 in negative ion mode. Samples were randomized before analysis. In addition, a quality control sample was injected after every 12th sample to monitor signal stability of the instrument.

### Data Processing and Identification of Metabolites

MS/MS spectra for polar metabolites were acquired on an Orbitrap ID-X Tribrid mass spectrometer (Thermo Scientific). A Vanquish Horizon UHPLC system with the same chromatographic conditions as described above, was interfaced with the mass spectrometer via electrospray ionization in both positive and negative mode with a spray voltage of 3.5 and 2.8 kV, respectively. The RF lens value was 35%. Data were acquired in data dependent acquisition (DDA) mode using the built-in deep scan option (AcquireX) with a mass range of 67-900 m/z. Features were automatically grouped based on adducts and isotopes. Unique features were used as an inclusion list. Peaks that were present in an extraction blank and not at least three times higher in the plasma samples were excluded. Features triggered for MS/MS in the first DDA run were then moved to the exclusion list for subsequent injections (total of 4) to fragment also lower abundant compounds. Inclusion and exclusion list were used with 5 ppm tolerance. MS1 scans were acquired at a resolution of 60K with an AGC target of 1e5 and a maximum injection time of 100 ms. An intensity threshold of 2e4 and a dynamic exclusion of 6 s was used. The isolation window was 1.5 Da and MS2 scans were acquired at 15K resolution. This workflow was performed on a pooled sample in both positive and negative polarity with different collision energies in the range of 20 to 50% for HCD and 30% for CID to maximize identifications.

MS/MS for polar metabolites as well as lipids were acquired using an iterative approach in the MassHunter Acquisition Software (Version 10.1.48, Agilent Technologies) on an Agilent 6540 or 6545 QTOF, respectively. The same source settings as for MS1 data acquisition were used. MS2 were acquired at a scan rate of 3 spectra/s with different intensity thresholds and collision energies of 10, 20, and 40 V to increase identification rates.

Polar metabolite identifications were supported by matching the retention time, accurate mass, and MS/MS fragmentation data to a library created from authentic reference standards (Mass Spectrometry Metabolite Library supplied by IROA Technologies, Millipore Sigma, St. Louis, MO, USA) and online MS/MS libraries (Human Metabolome Database (HMDB, https://hmdb.ca), Mass Bank of North America (MoNA, https://mona.fiehnlab.ucdavis.edu/), and mzCloud (https://mzcloud.org)). Lipid iterative MS/MS data were annotated with the Agilent Lipid Annotator software tool.

All data files were then analyzed in Skyline (Version 20.1.0.155). m/z values of the metabolite and lipid target lists obtained from the annotation workflow, which had at least a match to an online library, were extracted under consideration of retention times.

### Age Modelling Using Metabolomics

### Data Preprocessing

The Python library Pandas (Mckinney W., 2010. Proc. of the 9th Python in Science Conf. 56-61) was used for data cleaning, filtering, and transforming omics datasets to make them suitable for machine learning (ML) models. The relative abundance of metabolites from the 39 dog samples was provided for age prediction and feature importance assessment that identifies key biomarkers using ML regression ensemble models such as random forest and AdaBoost. After initial cleaning of the raw data, the dataset was transformed and scaled using min-max normalization method from the scikit learn (Fabian P et al., 2011. Journal of Machine Learning Research. 2825-2830) preprocessing package, Z-score, and log 2 normalization methods.

$$Min Ma x Scaler = \frac{value - min}{max - min} \qquad ( 1 )$$

$$Z - score = \frac{value - μ}{σ} \qquad ( 2 )$$

where μ is the mean value of the feature and σ is the standard deviation of the feature.

The dataset was then split into training and testing sets using the stratified random sampling method, which ensures that the class distribution is preserved in both sets. The 80-20 rule was used, which means that 80% of the data is allocated for training and 20% for testing.

### Regression Machine Learning Method: Age, Continuous Prediction

Scikit-learn 1.3.2 (Fabian, 2011) was used for ML workflow to predict the chronological age of dogs from the metabolomics dataset created from the early integration method. In this study, two ensemble learning methods, the Random Forest (RF) regressor and the AdaBoost (AB) regressor, were used for the regression task.

The regression model performance is highly affected by hyperparameters. The grid search cross-validation was used which is the algorithm for finding the optimal combination of the hyperparameters for the regression model. The grid search cross-validation involves defining a grid of possible values for each hyperparameter and evaluating the model performance on a validation set for each combination of hyperparameters. An example random forest parameter grid is:

```
param_grid = {
  ′bootstrap′: [True],
  ′max_depth′: [′None′, 1, 2, 3, 4, 5],
  ′max_features′: [′auto′, 1, 2, 3, 4, 5],
  ′min_samples_leaf′: [1, 2, 3, 4, 5],
  ′min_samples_split′: [2, 5, 10],
  ′n_estimators′: [10, 20, 100, 200]
}
```

The dataset was stratified according to two age groups, young and senior, to ensure both training and testing sets have an equal ratio of young and senior individuals. The random state number was set to 42 and the train_test_split function was utilized in the following manner.

$$X\_train , X\_test , y\_train , y\_test = train\_test \_split ( X , y , test\_size = 0.2 , stratify = age\_groups , random\_state = 42 ) \qquad ( 3 )$$

RandomForestRegressor Algorithm: Randomized search cross-validation is utilized to identify the optimal parameters for the RandomForest regression model. In the model, the number of estimators is set to 1200, max_depth is set to 3, and random_state number is set to 0.

$$regr = RandomForestRegressor ( n\_estimators = 1200 , min\_samples \_split = 10 , max\_features = ‘ sqrt ’ , max\_depth = 3 , bootstrap = False , random\_state = 0 ) \qquad ( 4 )$$

To ensure consistent results (i.e., r² value 0.39) when using the canine metabolomics dataset, all specified random_state values should be set to fixed numbers. The importance of features can be determined using the feature_importances_attribute. We can identify and select the top 30 metabolites from a pool of 501 and reconstruct the model using the same algorithm to achieve the final R-squared value of 0.8 with top 5 features.

### Metabolite Ranking

Accurate sequencing of metabolites is essential to maintain the current model's performance, which predicts the age of previously unseen data. The relative abundance values of metabolites are inputted as a NumPy array into the model (2) in the following rank order as shown in Table 1.

Classification Machine Learning Method: Young Vs. Senior, Categorical Prediction

The supervised ML classification was performed for classifying groups between young (under 7 years old) and senior dogs (above 7 years old) from the metabolomics dataset. The partial least square (PLS2—two dependent variables: young vs. senior) algorithm was used mainly for the dimensionality reduction and the feature extraction. Then, the support vector machine (SVM) was used as a classifier to separate the two groups (senior and young). The SVM model performance was evaluated using the accuracy and F1-score metrics from the confusion matrix on the test set. The middle-integration approach was performed using the DIABLO method in the mixOmics package (Le Cao, 2009) from R 4.2.2. The DIABLO method constructs latent components like PLS2 but the sum of covariance between all pairs of omics data sets is maximized.

### Results

### Continuous Age Model

With the metabolomics dataset, the Random Forest regressor showed the best performance (r² value: 0.39, AB: 0.18). A set of optimal hyperparameter values was obtained using grid search with 5-fold cross-validation list.

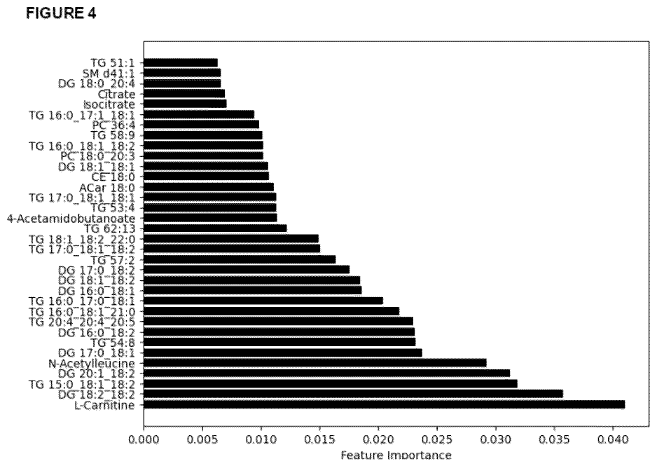

The top 35 metabolites, ranked in order of importance, for the canine continuous age model are shown in FIG. 4 and Table 4.

TABLE 4
The Random Forest regressor model identifies the significance
of each feature. L-Carnitine is the most important feature.

| | | Importance |
|---|---|---|
| Rank | Top Metabolites | Value |
| 1 | L-Carnitine | 0.041002 |
| 2 | DG 18:2_18:2 | 0.035766 |
| 3 | TG 15:0_18:1_18:2 | 0.031837 |
| 4 | DG 20:1_18:2 | 0.031255 |
| 5 | N-Acetylleucine | 0.029237 |
| 6 | DG 17:0_18:1 | 0.023747 |
| 7 | TG 54:8 | 0.023160 |
| 8 | DG 16:0_18:2 | 0.023100 |
| 9 | TG 20:4_20:4_20:5 | 0.022966 |
| 10 | TG 16:0_18:1_21:0 | 0.021770 |
| 11 | TG 16:0_17:0_18:1 | 0.020407 |
| 12 | DG 16:0_18:1 | 0.018625 |
| 13 | DG 18:1_18:2 | 0.018481 |
| 14 | DG 17:0_18:2 | 0.017539 |
| 15 | TG 57:2 | 0.016402 |
| 16 | TG 17:0_18:1_18:2 | 0.015067 |
| 17 | TG 18:1_18:2_22:0 | 0.014935 |
| 18 | TG 62:13 | 0.012234 |
| 19 | 4-Acetamidobutanoate | 0.011350 |
| 20 | TG 53:4 | 0.011341 |
| 21 | TG 17:0_18:1_18:1 | 0.011336 |
| 22 | ACar 18:0 | 0.011138 |
| 23 | CE 18:0 | 0.010660 |
| 24 | DG 18:1_18:1 | 0.010632 |
| 25 | PC 18:0_20:3 | 0.010208 |
| 26 | TG 16:0_18:1_18:2 | 0.010170 |
| 27 | TG 58:9 | 0.010122 |
| 28 | PC 36:4 | 0.009844 |
| 29 | TG 16:0_17:1_18:1 | 0.009464 |
| 30 | TG 54:8 | 0.008404 |
| 31 | Isocitrate | 0.007086 |
| 32 | Citrate | 0.006914 |
| 33 | DG 18:0_20:4 | 0.006590 |
| 34 | SM d41:1 | 0.006576 |
| 35 | TG 51:1 | 0.006304 |

### Continuous Age Models Exhibit High Performance

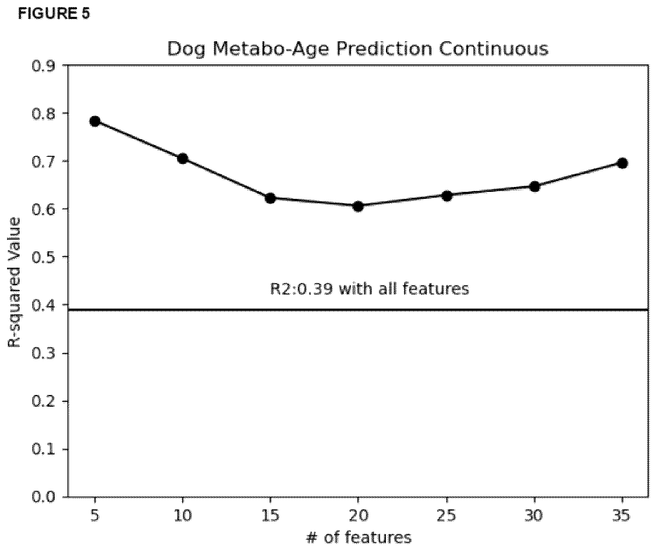

When reconstructing the model with the most important features, the model performance consistently achieves an r-squared value of greater than 0.6. The r-squared value was plotted as a function of the number of features as shown in FIG. 5. For example, the regressor model reconstructed with the top 5, top 10, and top 35 features show r-squared values of 0.79, 0.70 and 0.69, respectively.

Continuous Age Models with Randomly Selected Features from the Top 30 Features Exhibit High Performance

The models were reconstructed using 5 or 10 randomly selected features from the top 10 or top 30 features. The resulting r² values are displayed in Table 5.

TABLE 5
R-squared value from random forest regression model with the random
selection of 5 or 10 features from top 10 or top 30 metabolites

| | r² value |
|---|---|
| Five random features from top 10 | |
| L-Carnitine, N-Acetylleucine, DG 16:0_18:2, DG 17:0_18:1, TG | 0.38 |
| 15:0_18:1_18:2 | |
| TG 16:0_18:1_21:0, DG 16:0_18:2, DG 18:2_18:2, L-Carnitine, TG | 0.40 |
| 20:4_20:4_20:5 | |
| N-Acetylleucine, TG 16:0_18:1_21:0, TG 54:8, TG 15:0_18:1_18:2, TG | 0.55 |
| 20:4_20:4_20:5 | |
| DG 20:1_18:2, L-Carnitine, TG 15:0_18:1_18:2, N-Acetylleucine, DG | 0.43 |
| 18:2_18:2 | |
| Five Random Features from top 30 | |
| [DG 18:2_18:2, DG 20:1_18:2, TG 54:8, CE 18:0, DG 16:0_18:2] | 0.34 |
| [TG 18:1_18:2_22:0, DG 18:1_18:1, L-Carnitine, TG 17:0_18:1_18:1, TG | 0.32 |
| 16:0_17:0_18:1] | |
| [DG 17:0_18:2, DG 18:1_18:2, DG 18:2_18:2, TG 18:1_18:2_22:0, TG 54:8] | 0.34 |
| [DG 16:0_18:1, TG 16:0_17:0_18:1, TG 18:1_18:2_22:0, TG 62:13, TG | 0.40 |
| 53:4] | |
| Ten Random Features from top 30 | |
| [ACar 18:0, TG 16:0_17:1_18:1, TG 62:13, TG 54:8, TG 54:8, CE 18:0, PC | 0.41 |
| 18:0_20:3, TG 17:0_18:1_18:2, PC 36:4, N-Acetylleucine] | |
| [TG 16:0_18:1_18:2, TG 62:13, TG 58:9, DG 17:0_18:1, TG | 0.35 |
| 18:1_18:2_22:0, TG 54:8, TG 20:4_20:4_20:5, TG 17:0_18:1_18:2, PC | |
| 36:4, DG 16:0_18:1] | |
| [TG 15:0_18:1_18:2, TG 20:4_20:4_20:5, DG 17:0_18:1, TG 57:2, TG | 0.38 |
| 17:0_18:1_18:2, L-Carnitine, DG 18:2_18:2, TG 16:0_17:0_18:1, DG | |
| 18:1_18:1, TG 53:4] | |
| [PC 36:4, TG 62:13, DG 16:0_18:1, DG 18:1_18:2, TG 18:1_18:2_22:0, TG | 0.44 |
| 17:0_18:1_18:1, TG 17:0_18:1_18:2, TG 15:0_18:1_18:2, TG 54:8_255, TG | |
| 57:2] | |

### Ridge Regression Model Comparison

The Random Forest regression model's performance was compared with a linear regression model-ridge regression. The metabo-age was predicted based on the top 35 important features which were obtained from the Random Forest model. The ridge regression estimator was obtained by adding a penalty term to the ordinary least square (OLS) estimator. It aims to reduce the variance of coefficient estimates in multiple linear regression models and provides a way to handle multicollinearity by introducing a penalty term that shrinks the coefficient toward zero. The mathematical formula for the linear least squares with the regularization is as follows:

$${‖ y - {X}_{w} ‖}_{2}^{2} + alpha * {‖ w ‖}_{2}^{2} \qquad ( 5 )$$

where y represents the response variable, Xw is the feature matrix, alpha is the regularization parameter controlling the strength of the penalty, and w is the vector of regression coefficients.

Scikit-learn machine learning library was used to build the ridge regressor for this study,

$$ridge\_model = linear\_model . RidgeCV ( alpha = np . logspace ( - 6 , 6 , 13 ) ) \qquad ( 6 )$$

RidgeCV implements ridge regression with Leave-One-Out cross-validation of the alpha parameter.

Feature weight vectors were calculated by using the coef_attribute as follows:

$$ridge\_model . coef\_ \qquad ( 7 )$$

The linear regression—ridge regression—was performed using various numbers of features. Increasing numbers of features were used in the regression model in order of their importance as identified by the Random Forest model. The r² value was shown to vary depending on the number of features included in the regression model.

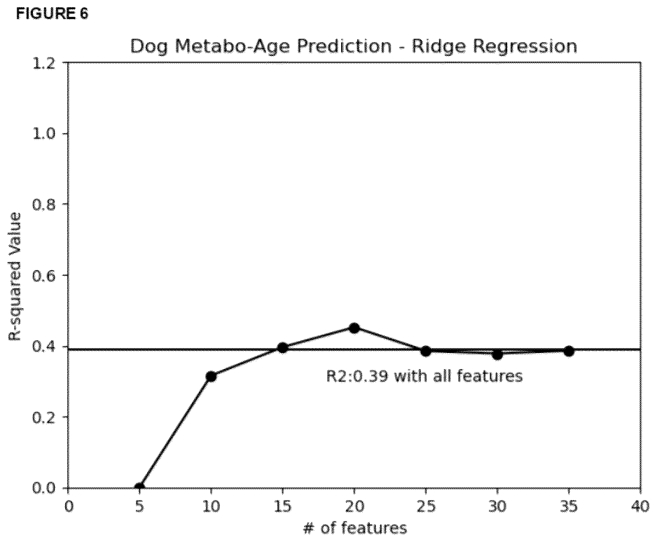

As is shown in FIG. 6, the r² value of the ridge regression model reaches a value of 0.48 when using the top 20 most important features and a value of 0.38 when using the top 35 most important features as identified using the Random Forest model. This is compared to the r² value of 0.39 reached by a Random Forest model using all features.

The feature weight vectors as determined by ridge regression for the top 35 features identified by the Random Forest model are provided in Table 6 below.

TABLE 6
Feature weight vectors as determined by ridge regression
for top 35 Random Forest model features.

| Feature | Weight Vector (w) |
|---|---|
| L-Carnitine | 2.959699 |
| DG 18:2_18:2 | 0.031054 |
| TG 15:0_18:1_18:2 | 0.370328 |
| DG 20:1_18:2 | 0.028449 |
| N-Acetylleucine | 1.003469 |
| DG 17:0_18:1 | −0.416991 |
| TG 54:8 | −1.273438 |
| DG 16:0_18:2 | 0.264034 |
| TG 20:4_20:4_20:5 | −0.931780 |
| TG 16:0_18:1_21:0 | 0.127301 |
| TG 16:0_17:0_18:1 | −0.202812 |
| DG 16:0_18:1 | 1.266777 |
| DG 18:1_18:2 | 0.175994 |
| DG 17:0_18:2 | 0.088146 |
| TG 57:2 | 0.067389 |
| TG 17:0_18:1_18:2 | 0.405904 |
| TG 18:1_18:2_22:0 | 0.870497 |
| TG 62:13 | −1.303469 |
| 4-Acetamidobutanoate | 0.742529 |
| TG 53:4 | 0.230246 |
| TG 17:0_18:1_18:1 | 0.117367 |
| ACar 18:0 | 0.711948 |
| CE 18:0 | 1.232873 |
| DG 18:1_18:1 | 0.749728 |
| PC 18:0_20:3 | 0.886624 |
| TG 16:0_18:1_18:2 | 0.977739 |
| TG 58:9 | 0.035293 |
| PC 36:4 | 2.334300 |
| TG 16:0_17:1_18:1 | 0.356104 |
| TG 54:8 | −0.565971 |
| Isocitrate | 1.286450 |
| Citrate | 1.069667 |
| DG 18:0_20:4 | 1.010641 |
| SM d41:1 | 0.824034 |
| TG 51:1 | 0.997848 |

A linear regression model was capable of achieving high r² values when using groups of features identified to have high importance by the Random Forest ML model.

Categorical Young Vs. Senior Age Model

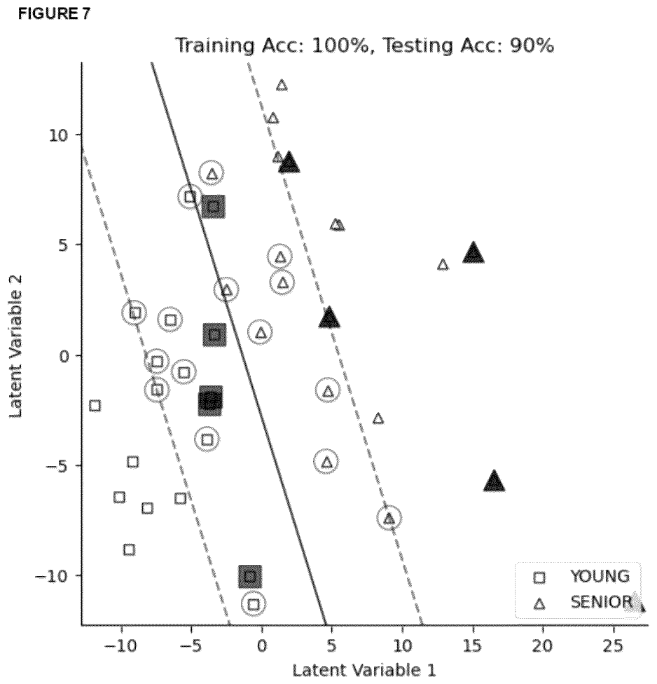

SVM classification was carried out on the metabolites to develop a model representing young vs senior canines was developed (FIG. 7). The top metabolites, in order of importance, for the canine young versus senior age classification model are shown in Table 3.

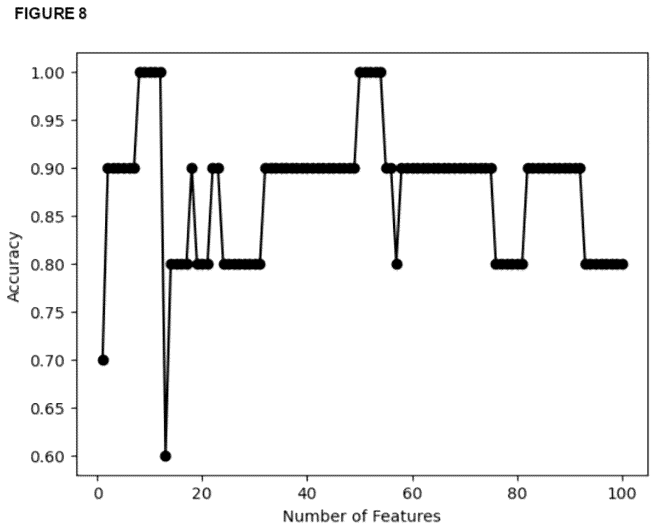

The accuracy of the canine young versus senior classification model was measured for models with varying numbers of the most important features included. The accuracy of the classification model as a function of the number of features is shown in FIG. 8 and Table 7.

TABLE 7
Top 60 metabolites - Dog Categorical Young vs. Senior Model

| Rank by | | ML Accuracy |
|---|---|---|
| P-value | Top Metabolites | by # of features |
| 1 | 1-Phenylethanol | 0.7 |
| 2 | 10-Hydroxydecanoate | 0.9 |
| 3 | DG 18:0_20:4 | 0.9 |
| 4 | DG 18:1_22:5 | 0.9 |
| 5 | TG 55:8 | 0.9 |
| 6 | DG 16:0_18:2 | 0.9 |
| 7 | DG 16:0_18:1 | 0.9 |
| 8 | DG 16:1_18:2 | 1.0 |
| 9 | TG 55:2 | 1.0 |
| 10 | TG 18:2_20:4_21:0 | 1.0 |
| 11 | HexCer_NS d18:1_16:0 | 1.0 |
| 12 | TG 51:1 | 1.0 |
| 13 | LPC 16:0/0:0 | 0.6 |
| 14 | TG 56:7 | 0.8 |
| 15 | N-Acetylputrescine | 0.8 |
| 16 | DG 18:1_20:4 | 0.8 |
| 17 | TG 58:11 | 0.8 |
| 18 | DG 17:1_18:2 | 0.9 |
| 19 | DG 17:0_18:2 | 0.8 |
| 20 | TG 50:6 | 0.8 |
| 21 | TG 16:0_18:2_18:2 | 0.8 |
| 22 | TG 52:8 | 0.9 |
| 23 | TG 50:1 | 0.9 |
| 24 | TG 56:6 | 0.8 |
| 25 | TG 50:4 | 0.8 |
| 26 | TG 52:6 | 0.8 |
| 27 | TG 52:5 | 0.8 |
| 28 | TG 48:6 | 0.8 |
| 29 | TG 18:0_18:1_18:1 | 0.8 |
| 30 | DG 18:0_22:0 | 0.8 |
| 31 | TG 16:0_16:0_18:2 | 0.8 |
| 32 | TG 57:2 | 0.9 |
| 33 | TG 18:0_18:1_18:2 | 0.9 |
| 34 | TG 46:2 | 0.9 |
| 35 | TG 58:9 | 0.9 |
| 36 | Leucine | 0.9 |
| 37 | TG 56:6 | 0.9 |
| 38 | TG 16:0_18:0_18:1 | 0.9 |
| 39 | TG 54:7 | 0.9 |
| 40 | TG 15:0_16:0_16:1 | 0.9 |
| 41 | TG 16:0_16:0_17:0 | 0.9 |
| 42 | TG 54:4 | 0.9 |
| 43 | Phenylalanine | 0.9 |
| 44 | TG 18:1_20:4_22:4 | 0.9 |
| 45 | TG 18:1_18:2_22:1 | 0.9 |
| 46 | TG 55:1 | 0.9 |
| 47 | PC 17:0_20:4 | 0.9 |
| 48 | TG 15:0_18:1_18:2 | 0.9 |
| 49 | TG 16:1_18:2_20:4 | 0.9 |
| 50 | Isoleucine | 1.0 |
| 51 | TG 16:0_18:2_20:4 | 1.0 |
| 52 | TG 48:3 | 1.0 |
| 53 | TG 50:5 | 1.0 |
| 54 | TG 56:5 | 1.0 |
| 55 | TG 18:1_18:2_21:0 | 0.9 |
| 56 | TG 16:0_17:0_18:0 | 0.9 |
| 57 | TG 48:5 | 0.8 |
| 58 | TG 14:0_16:0_20:4 | 0.9 |
| 59 | TG 50:7 | 0.9 |
| 60 | 4-Hydroxybenzoate | 0.9 |

The accuracy of the model is 0.90 when using the 2 most important features, and reaches 1.00 when using the 8 most important features.

The accuracy value of the SVM model was measured when using 5 or 10 randomly selected features from the top 10 or top 30 most important features. The accuracy of the models using these features is shown in Table 8.

TABLE 8
SVM model accuracy when using 5 or 10 randomly selected features
from the top 10 or top 30 most important features.

| | Accuracy (%) |
|---|---|
| Five Random Features from top 10 | |
| DG 16:0_18:2, 1-Phenylethanol, DG 16:0_18:1, TG 18:2_20:4_21:0, | 0.90 |
| TG 55:8 | |
| DG 16:1_18:2, 10-Hydroxydecanoate, DG 16:0_18:2, DG 18:0_20:4, | 0.72 |
| TG 18:2_20:4_21:0 | |
| TG 55:2, TG 55:8, TG 18:2_20:4_21:0, 1-Phenylethanol, 10- | 0.72 |
| Hydroxydecanoate | |
| 1-Phenylethanol, TG 55:8, TG 18:2_20:4_21:0, DG 16:1_18:2, DG | 0.93 |
| 16:0_18:2 | |
| TG 55:8, DG 16:1_18:2, DG 18:1_22:5, DG 16:0_18:1, DG 18:0_20:4 | 0.90 |
| DG 16:0_18:1, DG 18:1_22:5, 10-Hydroxydecanoate, TG 55:8, TG | 0.86 |
| 55:2 | |
| Five Random Features from top 30 | |
| TG 52:8, DG 16:1_18:2, TG 52:6, TG 56:7, DG 18:1_20:4 | 0.86 |
| TG 56:6, DG 16:0_18:1, 10-Hydroxydecanoate, HexCer_NS | 0.76 |
| d18:1_16:0, TG 52:5 | |
| LPC 16:0/0:0, DG 18:0_22:0, TG 52:8, TG 58:11, TG 18:2_20:4_21:0 | 0.90 |
| 1-Phenylethanol, HexCer_NS d18:1_16:0, TG 50:1, DG 16:1_18:2, | 0.66 |
| DG 18:0_22:0 | |
| TG 50:4, DG 16:0_18:1, TG 55:2, TG 56:7, DG 18:0_20:4 | 0.86 |
| TG 48:6, TG 18:0_18:1_18:1, DG 18:1_20:4, TG 16:0_18:2_18:2, TG | 0.90 |
| 52:6 | |
| Ten Random Features from top 30 | |
| TG 18:0_18:1_18:1, DG 17:0_18:2, TG 55:2, TG 50:1, DG 18:0, DG | 0.83 |
| 18:1_22:5, LPC 16:0/0:0, DG 17:1_18:2, DG 16:1_18:2, TG | |
| 18:2_20:4_21:0 | |
| DG 18:1_22:5, TG 52:5, DG 18:1_20:4, TG 51:1, TG 48:6_254, TG | 0.86 |
| 56:7, N-Acetylputrescine, DG 18:0_22:0, TG 16:0_18:2_18:2, TG | |
| 18:0_18:1_18:1 | |
| HexCer_NS d18:1_16:0, TG 55:2, DG 16:0_18:1, DG 18:1_20:4, 1- | 0.83 |
| Phenylethanol, 10-Hydroxydecanoate, TG 58:11, TG 52:5, TG | |
| 18:0_18:1_18:1, TG 50:4 | |
| TG 58:11, TG 52:6, LPC 16:0/0:0, TG 52:5, DG 17:0_18:2, TG 56:6, | 0.90 |
| TG 52:8, TG 18:2_20:4_21:0, 10-Hydroxydecanoate, TG 50:1 | |
| TG 50:4, TG 55:8, 1-Phenylethanol, TG 50:1, TG 18:0_18:1_18:1, | 0.79 |
| N-Acetylputrescine, DG 16:0_18:1_37, TG 52:6_224, TG 56:7, 10- | |
| Hydroxydecanoate | |
| TG 55:8, HexCer_NS d18:1_16:0, ′TG 52:6, TG 18:0_18:1_18:1, DG | 0.83 |
| 17:1_18:2, TG 48:6, TG 50:1, N-Acetylputrescine, TG 50:4, TG | |
| 16:0_18:2_18:2 | |

TABLE 1
Biomarker

L-Carnitine
DG 18:2_18:2
TG 15:0_18:1_18:2
DG 20:1_18:2
N-Acetylleucine
DG 17:0_18:1
TG 54:8
DG 16:0_18:2
TG 20:4_20:4_20:5
TG 16:0_18:1_21:0
TG 16:0_17:0_18:1
DG 16:0_18:1
DG 18:1_18:2
DG 17:0_18:2
TG 57:2
TG 17:0_18:1_18:2
TG 18:1_18:2_22:0
TG 62:13
4-Acetamidobutanoate
TG 53:4
TG 17:0_18:1_18:1
ACar 18:0
CE 18:0
DG 18:1_18:1
PC 18:0_20:3
TG 16:0_18:1_18:2
TG 58:9
PC 36:4
TG 16:0_17:1_18:1
1-Phenylethanol
10-Hydroxydecanoate
DG 18:0_20:4
DG 18:1_22:5
TG 55:8
DG 16:1_18:2
TG 55:2
TG 18:2_20:4_21:0
HexCer_NS d18:1_16:0
TG 51:1
LPC 16:0/0:0
TG 56:7
N-Acetylputrescine
DG 18:1_20:4
TG 58:11
DG 17:1_18:2
TG 50:6
TG 16:0_18:2_18:2
TG 52:8
TG 50:1
TG 56:6
TG 50:4
TG 52:6
TG 52:5
TG 48:6
TG 18:0_18:1_18:1
DG 18:0_22:0
Isocitrate
Citrate
DG 18:0_20:4
SM d41:1
TG 51:1
TG 16:0_16:0_18:2
TG 18:0_18:1_18:2
TG 46:2
Leucine
TG 16:0_18:0_18:1
TG 54:7
TG 15:0_16:0_16:1
TG 16:0_16:0_17:0
TG 54:4
Phenylalanine
TG 18:1_20:4_22:4
TG 18:1_18:2_22:1
TG 55:1
PC 17:0_20:4
G 15:0_18:1_18:2
TG 16:1_18:2_20:4
Isoleucine
TG 16:0_18:2_20:4
TG 48:3
TG 50:5
TG 56:5
TG 18:1_18:2_21:0
TG 16:0_17:0_18:0
TG 48:5
TG 14:0_16:0_20:4
TG 50:7
4-Hydroxybenzoate

TABLE 2

| Rank | Biomarker |
|---|---|
| 1 | L-Carnitine |
| 2 | DG 18:2_18:2 |
| 3 | TG 15:0_18:1_18:2 |
| 4 | DG 20:1_18:2 |
| 5 | N-Acetylleucine |
| 6 | DG 17:0_18:1 |
| 7 | TG 54:8 |
| 8 | DG 16:0_18:2 |
| 9 | TG 20:4_20:4_20:5 |
| 10 | TG 16:0_18:1_21:0 |
| 11 | TG 16:0_17:0_18:1 |
| 12 | DG 16:0_18:1 |
| 13 | DG 18:1_18:2 |
| 14 | DG 17:0_18:2 |
| 15 | TG 57:2 |
| 16 | TG 17:0_18:1_18:2 |
| 17 | TG 18:1_18:2_22:0 |
| 18 | TG 62:13 |
| 19 | 4-Acetamidobutanoate |
| 20 | TG 53:4 |
| 21 | TG 17:0_18:1_18:1 |
| 22 | ACar 18:0 |
| 23 | CE 18:0 |
| 24 | DG 18:1_18:1 |
| 25 | PC 18:0_20:3 |
| 26 | TG 16:0_18:1_18:2 |
| 27 | TG 58:9 |
| 28 | PC 36:4 |
| 29 | TG 16:0_17:1_18:1 |
| 30 | TG 54:8 |
| 31 | Isocitrate |
| 32 | Citrate |
| 33 | DG 18:0_20:4 |
| 34 | SM d41:1 |
| 35 | TG 51:1 |

TABLE 3

| Rank | Biomarker |
|---|---|
| 1 | 1-Phenylethanol |
| 2 | 10-Hydroxydecanoate |
| 3 | DG 18:0_20:4 |
| 4 | DG 18:1_22:5 |
| 5 | TG 55:8 |
| 6 | DG 16:0_18:2 |
| 7 | DG 16:0_18:1 |
| 8 | DG 16:1_18:2 |
| 9 | TG 55:2 |
| 10 | TG 18:2_20:4_21:0 |
| 11 | HexCer_NS d18:1_16:0 |
| 12 | TG 51:1 |
| 13 | LPC 16:0/0:0 |
| 14 | TG 56:7 |
| 15 | N-Acetylputrescine |
| 16 | DG 18:1_20:4 |
| 17 | TG 58:11 |
| 18 | DG 17:1_18:2 |
| 19 | DG 17:0_18:2 |
| 20 | TG 50:6 |
| 21 | TG 16:0_18:2_18:2 |
| 22 | TG 52:8 |
| 23 | TG 50:1 |
| 24 | TG 56:6 |
| 25 | TG 50:4 |
| 26 | TG 52:6 |
| 27 | TG 52:5 |
| 28 | TG 48:6 |
| 29 | TG 18:0_18:1_18:1 |
| 30 | DG 18:0_22:0 |
| 31 | TG 16:0_16:0_18:2 |
| 32 | TG 57:2 |
| 33 | TG 18:0_18:1_18:2 |
| 34 | TG 46:2 |
| 35 | TG 58:9 |
| 36 | Leucine |
| 37 | TG 56:6 |
| 38 | TG 16:0_18:0_18:1 |
| 39 | TG 54:7 |
| 40 | TG 15:0_16:0_16:1 |
| 41 | TG 16:0_16:0_17:0 |
| 42 | TG 54:4 |
| 43 | Phenylalanine |
| 44 | TG 18:1_20:4_22:4 |
| 45 | TG 18:1_18:2_22:1 |
| 46 | TG 55:1 |
| 47 | PC 17:0_20:4 |
| 48 | G 15:0_18:1_18:2 |
| 49 | TG 16:1_18:2_20:4 |
| 50 | Isoleucine |
| 51 | TG 16:0_18:2_20:4 |
| 52 | TG 48:3 |
| 53 | TG 50:5 |
| 54 | TG 56:5 |
| 55 | TG 18:1_18:2_21:0 |
| 56 | TG 16:0_17:0_18:0 |
| 57 | TG 48:5 |
| 58 | TG 14:0_16:0_20:4 |
| 59 | TG 50:7 |
| 60 | 4-Hydroxybenzoate |

1. A method for determining the biological age of an animal, wherein the method comprises determining the level at least 1 biomarker in said animal, wherein said biomarker is selected from the biomarkers as listed in Table 1.

2. The method according to claim 1, wherein the levels of five or more biomarkers are determined in said animal, preferably wherein at least 2, at least 3, at least 4, at least 5, at least 10, at least 25, at least 50 or at least 75 of the biomarkers are selected from the biomarkers as listed in Table 1.

3. The method according to claim 1, wherein at least 1, at least 2, at least 3, or at least 4 biomarkers are selected from the biomarkers listed as biomarkers 1 to 5 in Table 2.

4. The method according to claim 1, wherein at least 5, at least 6, at least 7, at least 8 or at least 9 biomarkers are selected from the biomarkers listed as biomarkers 1 to 10 in Table 2.

5. The method according to claim 1, wherein at least 1, at least 2, at least 3, or at least 4 biomarkers are selected from the biomarkers listed as biomarkers 1 to 5 in Table 3.

6. The method according to claim 1, wherein the animal is a canine.

7. The method according to claim 1, wherein the determined levels of the biomarkers are the levels of the biomarkers in a sample obtained from the animal, wherein the sample is a tissue sample, a blood sample, a serum sample, or a plasma sample.

8. The method according to claim 1, wherein the method determines the numerical biological age of the animal.

9. The method according to claim 1, wherein the method determines the categorical biological age of the animal.

10. The method according to claim 9, wherein the categorical biological age may be junior, adult or senior.

11. The method according to claim 1, wherein the method further comprises comparing the biological age of the animal with the chronological age of the animal.

12. A method for selecting a dietary, pharmacological or lifestyle regime for an animal, the method comprising:
(i) determining the biological age of the animal by the method according to claim 1,
(ii) selecting a suitable dietary, pharmacological or lifestyle regime for the animal based on the biological age of the animal as determined in step (i).

13. The method according to claim 12, wherein the method further comprises comparing the biological age of the animal with the chronological age of the animal.

14. The method according to claim 13, wherein the suitable dietary, pharmacological or lifestyle regimen is selected based on the comparison of the determined biological age relative to the chronological age of the animal.

15. A method for determining the efficacy of a dietary, pharmacological or lifestyle regime for improving the biological age of an animal, the method comprising:
(i) determining the biological age of the animal by the method according to claim 1,
wherein the biological age is determined from a sample obtained from the animal before the dietary, pharmacological or lifestyle regime,
(ii) determining the biological age of the animal by the method according to claim 1,
wherein the biological age is determined from a sample obtained from the animal after the dietary, pharmacological or lifestyle regime, and
(iii) determining if there has been a change in the biological age of the animal between the sample obtained from the animal before and after the dietary, pharmacological or lifestyle regime has been applied.
